(19) United States
(12) Patent Application Publication
(10) Pub. No.: US 2012/0259168 A1
(43) Pub. Date: Oct. 11, 2012

(54) SYSTEMS AND METHODS FOR TREATING URINARY INCONTINENCE

(76) Inventors: Roger P. GOLDBERG, Evanston, IL (US); Douglas S. SCHERR, Scarsdale, NY (US)

(21) Appl. No.: 13/412,225

(22) Filed: Mar. 5, 2012

Related U.S. Application Data
(60) Provisional application No. 61/593,353, filed on Feb. 1, 2012.
(60) Provisional application No. 61/449,078, filed on Mar. 3, 2011.

(51) Int. Cl. A61F 2/04

(52) U.S. Cl. 600/30

(57) ABSTRACT

A surgical implant and a surgical apparatus for implanting the implant into a subject. The apparatus includes an insertion needle, a retainer fitted over the needle, and a handle for manipulating the needle. The implant includes a mesh sling, a distal anchor, and a proximal anchor, with the mesh held to the needle by the retainer during implantation. In use, the needle is inserted into the subject, the distal and proximal anchors are set, and the retainer is removed to deploy the mesh into place. A tensioning assembly can be manipulated to tension the mesh. In some embodiments, a lumen in the needle is used to deliver an anesthetic to the surgical site and/or an epoxy to the mesh to form anchors.

### CROSS-REFERENCE TO RELATED APPLICATIONS

This application claims the priority benefit of U.S. Provisional Patent Application Ser. No. 61/449,078, filed Mar. 3, 2011, and U.S. Provisional Patent Application Ser. No. 61/593,353, filed Feb. 1, 2012, which are hereby incorporated herein by reference.

### TECHNICAL FIELD

The present invention relates generally to the field of medical devices and procedures, and in particular to surgical systems and methods for treating urinary incontinence.

### BACKGROUND

Urinary incontinence is a medical condition in which the patient experiences the involuntary leakage of urine. This can be a very distressing problem that can negatively impact the quality of the patient's life. To date, known treatment methods have not proven entirely satisfactory. Accordingly, needs exist for improved systems and methods for treating urinary incontinence. It is to the provision of solutions to these and other problems that the present invention is primarily directed.

### SUMMARY

Generally described, the present invention relates to systems and methods for treating medical conditions such as urinary incontinence. The systems include a surgical implant and a surgical apparatus for implanting the implant into a subject. The apparatus includes an insertion needle, a retainer fitted over the needle, and a handle for manipulating the needle. The implant includes a mesh sling, a distal anchor, and a proximal anchor, with the mesh held to the needle by the retainer during implantation. In use, the needle is inserted into the subject, the distal and proximal anchors are set, and the retainer is removed to deploy the mesh into place. A tensioning string extending from the mesh and routed around the distal anchor can be manipulated to tension the mesh, and unidirectional mechanical stops on the tensioning string can be engaged to lock the mesh in the tensioned state. In other embodiments, a rotational element of the distal anchor is rotates with the needle to proximally reposition the distal anchor barbs to distally reposition the distal end fo the mesh and thereby tension the mesh. In some embodiments, a lumen in the needle is used to deliver an anesthetic to the surgical site and/or an epoxy to the mesh for forming anchors.

The specific techniques and structures employed to improve over the drawbacks of the prior systems and methods, and to accomplish the advantages described herein, will become apparent from the following detailed description of example embodiments and the appended drawings and claims.

### BRIEF DESCRIPTION OF THE DRAWINGS

FIG. 1 is a perspective view of a surgical system according to a first example embodiment of the present invention, showing an implant and a device for implanting the implant.

FIG. 2 is a perspective view of the implant of FIG. 1 in a deployed position.

FIG. 3 is a side view of a distal portion of the implant of FIG. 2.

FIG. 4 is a perspective view of a proximal portion of the implant of FIG. 2.

FIG. 5 is a perspective view of the device and implant of FIG. 1, showing the implant inserted into a subject's body according to an example method of use.

FIG. 6 shows the device and implant of FIG. 5 with the retainer removed to deploy the mesh.

FIG. 7 shows the device and implant of FIG. 6 with the mesh tensioned.

FIG. 8 is a side view of the upper portion of the implant of FIG. 6 with the mesh untensioned.

FIG. 9 shows the upper portion of the implant of FIG. 8 with the mesh being tensioned.

FIG. 10 is a right side detail view of an upper portion of the implant of FIG. 9 with the mesh being tensioned.

FIG. 11 shows the implant of FIG. 7 with the device removed.

FIG. 12 shows the implant of FIG. 11 with the excess suture removed.

FIG. 13 shows the implant of FIG. 12 with the proximal anchor removed.

FIG. 14 shows the implant of FIG. 13 implanted for long-term use.

FIG. 15 is a perspective view of a surgical system according to a second example embodiment of the present invention, showing an implant and a device for implanting the implant.

FIG. 16 is a side view of the surgical system of FIG. 15.

FIG. 17 is a perspective view of the implant of FIG. 15 in a deployed position.

FIG. 18 is a perspective view of a proximal portion of the implant of FIG. 17.

FIG. 19 is a perspective view of the device and implant of FIG. 15, showing the implant inserted into a subject's body according to an example method of use.

FIG. 20 shows the device and implant of FIG. 19 with the retainer removed to deploy the mesh.

FIG. 21 shows the device and implant of FIG. 20 with the mesh tensioned.

FIG. 22 shows the implant of FIG. 21 implanted for long-term use.

FIG. 23 is a perspective view of a distal anchor of a surgical system according to an alternative embodiment.

FIG. 24 is a perspective view of a surgical system according to a third example embodiment of the present invention, showing an implant and a device for implanting the implant.

FIG. 25 is a perspective view of a surgical system according to an alternative embodiment.

FIG. 26 is a perspective view of an implant of a surgical system according to a fourth example embodiment of the present invention.

FIG. 27 is a side view of the device and implant of FIG. 26, showing the implant inserted into a subject's body and the retainer being removed to deploy the mesh.

FIG. 28 is a perspective view showing the device and implant of FIG. 27 with the mesh being deployed.

FIG. 29 shows the implant of FIG. 28 with the base of the mesh being further deployed.

FIG. 30 is a perspective view of a mesh of a surgical system according to an alternative embodiment.

FIG. 31 is a perspective view of an implant of a surgical system according to a fifth example embodiment of the present invention, showing the mesh base deployed.

FIG. 32 is a side view of the implant of FIG. 31.

FIG. 33 is a perspective view of an implant of a surgical system according to a sixth example embodiment of the present invention, showing the mesh base deployed.

FIG. 34 is a side view of the implant of FIG. 33.

FIG. 35 is a perspective view of an implant of a surgical system according to a seventh example embodiment of the present invention, showing the mesh base deployed.

FIG. 36 is a side view of the implant of FIG. 35.

FIG. 37 is a perspective view of a surgical system according to an eighth example embodiment of the present invention, showing an implant and a device for implanting the implant.

FIG. 38 is a perspective view of the device and implant of FIG. 37, showing the implant inserted into a subject's body according to an example method of use.

FIG. 39 shows the device and implant of FIG. 38 with the retainer removed and the mesh and its base deployed.

FIG. 40 shows the device and implant of FIG. 39 with the mesh being tensioned.

FIG. 41 shows the device and implant of FIG. 40 with the mesh being tensioned by distally advancing the needle according to a first option.

FIG. 42 shows the device and implant of FIG. 41 with the mesh fully tensioned.

FIG. 43 shows the implant of FIG. 42 with the device removed.

FIG. 44 shows the implant of FIG. 43 with the excess suture removed.

FIG. 45 shows the implant and device of FIG. 40 with the mesh being tensioned according to a second option.

FIG. 46 shows the implant of FIG. 45 with the device removed and the mesh being tensioned by distally advancing the anchor-plate.

FIG. 47 shows the implant and device of FIG. 46 with the device removed.

FIG. 48 shows the implant and device of FIG. 47 with the excess suture removed.

FIG. 49 is a side view of a surgical system according to a ninth example embodiment of the present invention, showing an implant and a device for implanting the implant.

FIG. 50 is a perspective view of a handle and needle of the device of FIG. 49.

FIG. 51 is a perspective view of a glue dispenser of the device of FIG. 49.

FIG. 52 is a perspective view of a retainer tube of the device of FIG. 49.

FIG. 53 is a side view of the implant of FIG. 49 in a deployed position.

FIG. 54 is a perspective view of a distal anchor and distal end of the implant of FIG. 53.

FIG. 55 is a perspective view of a rotary element of the distal anchor of FIG. 53.

FIG. 56 is a perspective view of a securing element of the distal anchor of FIG. 53.

FIG. 57 is a side view of the device and implant of FIG. 49 ready for implantation according to an example method of use.

FIG. 58 is a perspective view of the device and implant of FIG. 57, showing the implant inserted into a subject's body.

FIG. 59 shows the device and implant of FIG. 58 with the retainer being removed and the distal anchor deploying.

FIG. 60 is a side view of the device and implant of FIG. 59, showing the retainer removed and the mesh deployed.

FIG. 61 shows the device and implant of FIG. 60 with the glue dispenser being actuated.

FIG. 62 shows the implant of FIG. 61 with glue dispensed onto the mesh.

FIG. 63 shows the implant of FIG. 62 with glue spots formed on the mesh.

FIG. 64 shows the implant of FIG. 63 with the mesh being tensioned by rotating the handle and needle.

FIG. 65 shows the implant of FIG. 64 with the mesh tensioned.

FIG. 66 shows the implant of FIG. 65 with the device being removed by reverse-rotating the handle and needle.

FIG. 67 shows the implant of FIG. 66 implanted for long-term use.

FIG. 68 is a perspective view of a surgical system according to a tenth example embodiment of the present invention, showing an implant and a device for implanting the implant.

FIG. 69 is a perspective view of a connector shaft, distal anchor, and proximal anchor of the implant of FIG. 68.

FIG. 70 shows the connector shaft, distal anchor, and proximal anchor of the implant of FIG. 68 assembled together.

FIG. 71 is a side view of the proximal anchor and a proximal portion of the connector shaft of the implant of FIG. 68.

FIG. 72 is a side view of the device and implant of FIG. 68 ready for implantation according to an example method of use.

FIG. 73 is a perspective view of the device and implant of FIG. 72, showing the implant inserted into a subject's body.

FIG. 74 is a side view of the device and implant of FIG. 73, showing the retainer being removed and the distal anchor being deployed.

FIG. 75 shows the device and implant of FIG. 74 with the retainer removed and the mesh and proximal anchor being deployed.

FIG. 76 shows the device and implant of FIG. 75 with the mesh being tensioned by rotating the handle and needle.

FIG. 77 shows the implant of FIG. 76 with the mesh tensioned.

FIG. 78 shows the implant of FIG. 77 with the device removed.

FIG. 79 shows the implant of FIG. 78 implanted for long-term use.

FIG. 80 is a perspective view of a surgical system according to an eleventh example embodiment of the present invention, showing an implant and a device for implanting the implant, and shown with a syringe for delivering anesthesia.

FIG. 81 is a perspective view of surgical system of FIG. 80, showing a glue dispenser with a cutaway portion.

FIG. 82 is a perspective view of the glue dispenser of FIG. 81.

FIG. 83 is a side view of a distal portion of the glue dispenser of FIG. 81.

FIG. 84 is a side view of a proximal portion of the glue dispenser of FIG. 81.

FIG. 85 is a side view of a securing element of the distal anchor of the implant of FIG. 80.

FIG. 86 is a perspective view of the securing element of FIG. 85.

FIG. 87 is a perspective view of the device and implant of FIG. 80 with the retainer removed.

FIG. 88 shows a distal portion of the implant of FIG. 87.

FIG. 89 shows a detail view of a portion of the mesh of FIG. 88.

FIG. 90 is a perspective view of a mesh of an implant according to an alternative embodiment.

FIG. 91 shows a detail view of a portion of the mesh of FIG. 88.

FIG. 92 is a perspective view of a portion of a mesh of an implant according to another alternative embodiment.

FIG. 93 shows a detail view of a portion of the mesh of FIG. 92.

### DETAILED DESCRIPTION OF EXAMPLE EMBODIMENTS

Generally described, the present invention relates to systems and methods for treating medical conditions such as urinary incontinence. The systems each include a surgical implant and a surgical apparatus/device/implement for implanting the surgical implant into a human or other animal subject. And the methods each include surgical procedures for using a surgical apparatus to implant a surgical implant into a human or other animal subject. While the implant and apparatus, and their method of use, are described herein for use in treating urinary incontinence in female humans, persons of ordinary skill in the art will appreciate how to adapt them for use in surgically treating the same or other medical conditions in males and/or other animals.

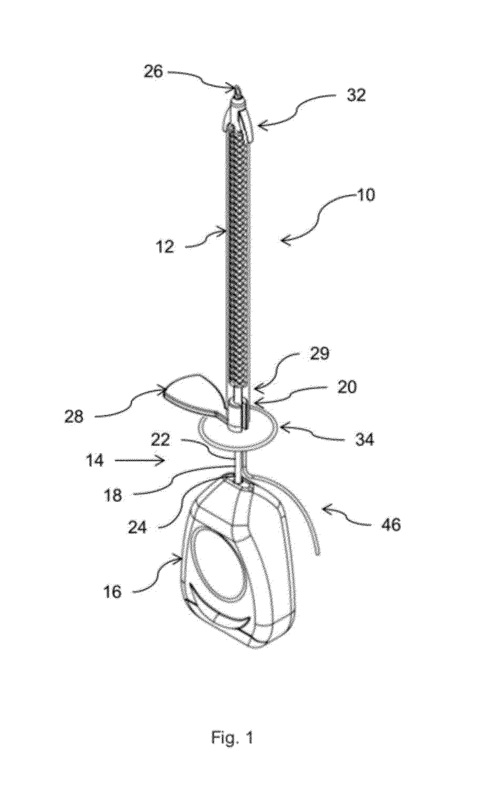

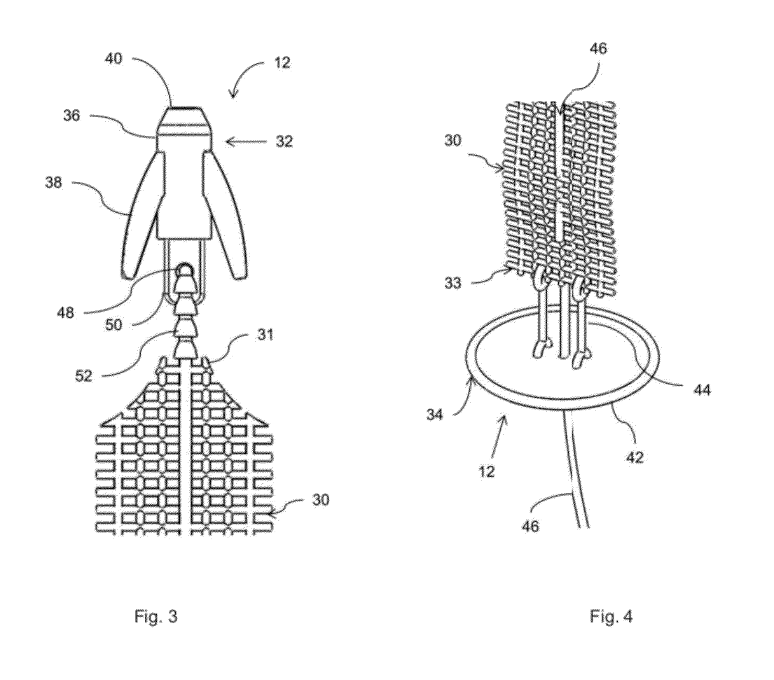

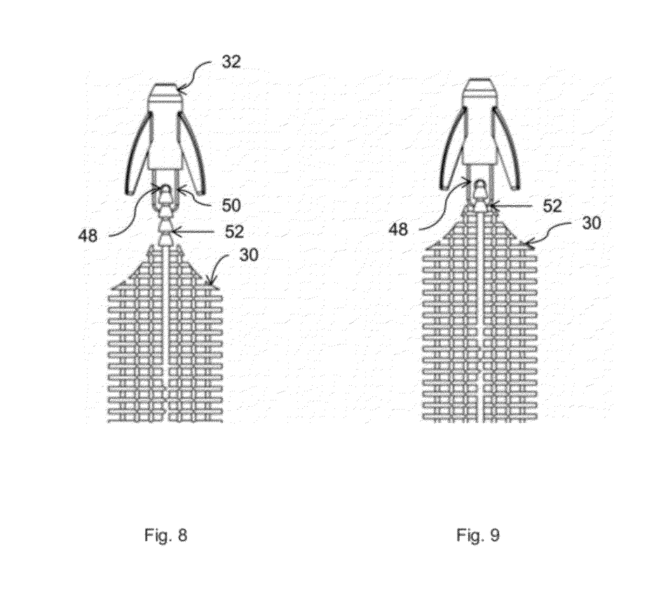

FIGS. 1-14 show a surgical system 10 according to a first example embodiment of the present invention, as well as a method of using the surgical system. The surgical system 10 includes a surgical implant 12 and a surgical apparatus 14 for implanting the surgical implant into a subject's body.

Referring primarily to FIG. 1, the surgical apparatus 14 includes a handle 16, a needle 18 extending from the handle, and an outer retainer 20 for holding the implant 12 in place on the needle. The handle 16 typically is sized, shaped, and otherwise configured for ease of manual grasping and manipulation in one hand of the surgical user. In some embodiments, the handle 16 has a hollow portion (e.g., with a flexible liner) that stores a fluid (e.g., epoxy, saline or another wash, or anesthetic or another medication) for dispensing (e.g., through a lumen of the needle 18, and actuated, e.g., by depressing a button on the handle to direct the fluid in to the needle under compression) into the subject. The handle 16 can be made of a plastic, metal, composite, or other material using fabrication techniques well known in the art.

The needle 18 includes an elongated shaft 22 with a proximal end 24 extending from the handle 16 and a sharp distal end 26 for puncturing tissue. In some embodiments, the needle 18 has a lumen formed within it that dispenses a fluid (e.g., epoxy, saline or another wash, or anesthetic or another medication, that is stored, e.g., in the handle 16) into the subject. The needle 18 can be made of a metal, plastic, composite, or other material using fabrication techniques well known in the art.

And the outer retainer 20 is positioned on the needle 18 holding the implant to the needle for implanting, and is removable once the needle is inserted into place so that the implant can be deployed from the retainer-less needle into place within the subject's body. The retainer 20 can include a detachment member 28 that is accessible by the user for manipulation/operation, with the needle 18 inserted into position, to remove the retainer and thereby deploy the implant 12 into place within the subject's body. The retainer 20 can be made of a plastic (e.g., a clear soft vinyl), metal, composite, or other material using fabrication techniques well known in the art.

In the depicted embodiment, for example, the retainer 20 is provided by a tube that substantially extends the length of and covers the implant 12 (or at least a mesh and proximal anchor thereof), has a longitudinal failure zone 29 extending along its length (see also FIG. 52), and is made of a resiliently deflectable material. In the depicted embodiment, the retainer tube 20 is made of a generally clear polymer so that the otherwise concealed underlying implant 12 is visible therethrough when the retainer is fitted on the needle 18.) The detachment member 28 is provided by a pull tab extending generally radially outward from the proximal end portion of the tube 20. The failure zone 29 can be provided by a preformed slit, a perforated or score line that fails upon application of a force to the detachment member, or another type of longitudinal failure zone that permits the tube 20 to be manipulated to deflect from a retaining closed position to a releasing open position. With the retainer tube 20 mounted onto the needle 18 over the implant 12, the tube is resiliently biased radially inward in the retaining closed position to retain the implant carried by the needle for insertion into the subject's body. And with the needle 18 inserted into the subject's body, the retainer tube 20 can be manipulated to resiliently deflect into the releasing open position for removal from the needle and the implant 12 in order to deploy and leave the implant in the subject's body. For example, the retainer tube 20 can be so manipulated to resiliently deflect into the releasing open position by pulling on the pull-tab detachment member 28 to pull the tube open along the failure zone 29 and to pull/slide it down off of the needle 18.

In other embodiments, the retainer is provided by a sheath, cage, coil, scroll, clip, clamp, clasp, telescopic assembly, fan-blade assembly, or other structure or assembly adapted to provide the implant retaining and releasing/deploying functionality described herein. In some such embodiments, the failure zone and detachment member need not be provided for the retainer to perform its intended function as described herein. And the detachment member in other embodiments is provided by an axially extending pull tab or string, a rotary retraction element, or another structure or assembly adapted to provide the implant retaining and deploying functionality described herein.

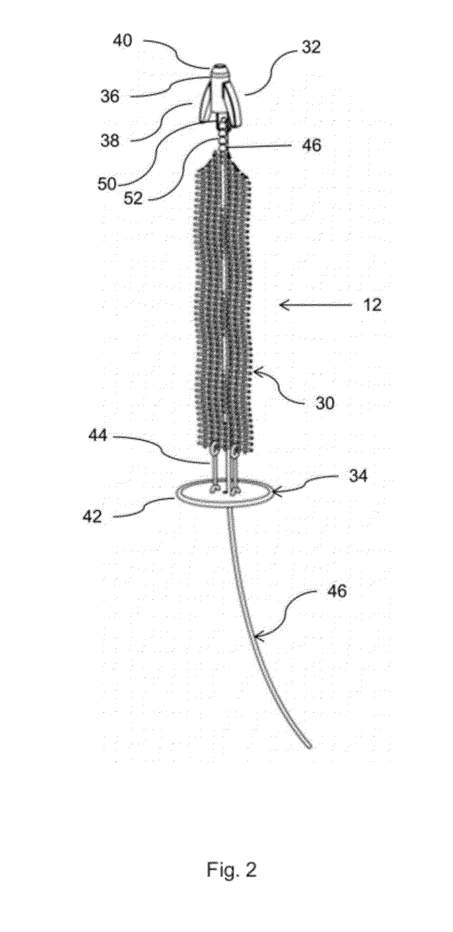

Referring primarily to FIGS. 2-4, the surgical implant 12 includes a surgical mesh sling and one or more anchors for securing the implanted mesh in place within the subject's body. In the depicted embodiment, for example, the surgical implant 12 includes a surgical mesh 30, a distal anchor 32, a proximal anchor 34, and a mesh-tensioning assembly 36 for tensioning the implanted mesh between the distal and proximal anchors. In other embodiments, only a distal or a proximal anchor is provided as an integral part of the implant 12, with the other end of the mesh 30 secured in place for example by separately provided sutures or epoxy (which are considered anchors for the purposes of this invention). And in other embodiments, the mesh tensioning is provided for example by implanting the mesh in a pre-tensioned state or by pulling on the proximal end of the mesh, without the implant or apparatus including a tensioning assembly for the mesh.

In the depicted embodiment, the surgical mesh 30 is an elongated, flexible, sheet-like mesh or latticed screen made of a resiliently flexible biocompatible material such as polypropylene. In other embodiments, the mesh is not a true mesh or latticed screen but rather a sheet, panel, or strip of material, which can be solid, perforated, woven, or otherwise configured and made for the intended sling use as described herein. In a typical commercial embodiment, the mesh 30 is about 40 mm long, though it can be longer or shorter as may be desired. The surgical mesh 30 is housed by the apparatus 14 (e.g., between the outer tube retainer 20 and the needle shaft 22) in a stored position ready for insertion into the subject's body. For example, in the stored position the mesh 30 can be collapsed (e.g., folded or wrapped) into a compact longitudinal arrangement around the needle 18 and retained there by the tube retainer 20 that is fitted over it. And upon the removal of the tube retainer 20, the mesh 30 is extended laterally outward (e.g., by the resiliency of the material causing it to unfurl) into a deployed position for use. In embodiments in which the mesh 30 is made of a material that is not sufficiently resilient for full self-deployment, the mesh can be manually pulled laterally outward into the deployed position by the practitioner. In some embodiments, the surgical mesh (or at least portions thereof) is made of a bio-absorbable material (e.g., of the type used in conventional bio-absorbable sutures).

The distal anchor 32 is positioned at and coupled to the distal end 31 of the mesh 30 and the proximal anchor 34 is positioned at and coupled to the proximal end 33 of the mesh. In the depicted embodiment, the distal anchor 32 has a body 36 and one or more (e.g., two, as shown) barbs 38 extending laterally from the body. The barbs 38 hook into the tissue to secure the distal anchor 32 in place. In the depicted embodiment, the barbs 38 are positioned distally of the distal end of the retainer 20 in the retaining closed position, and thus are not retained in a radially inward position during needle 18 and implant 12 insertion (though they typically deflect inward some upon insertion through a smaller-dimension surgical incision) and deployed radially outward upon removal of the retainer. In other embodiments, the barbs are positioned proximally of the distal end of the retainer in the retaining closed position, and thus they are retained in a radially inward position during needle and implant insertion and then resiliently deflected and deployed radially outward upon removal of the retainer. And the distal-anchor body 36 includes a receiver 40 (e.g., a center aperture as depicted, or a laterally positioned aperture or notch) that is engaged by the needle 18 so that when inserting the needle into the subject's body the distal anchor 32 is carried by the needle to the implantation site for anchoring.

The proximal anchor 34 of the depicted embodiment is provided by a plate (e.g., a disc or other-shaped plate with smooth edges) 42 that is positioned at and coupled to the proximal end of the mesh 30. Typically, the anchor-disc 42 is made of a biocompatible material such as plastic and secured to the proximal end of the mesh 30 with one or more mesh-to-anchor connectors 44. The mesh-to-anchor connectors 44 can be provided by conventional sutures, strings, cords, ties, or other conventional connecting elements for coupling the mesh 30 to the proximal anchor 34. For example, in the depicted embodiment, the anchor-disc 42 is secured to the mesh 30 with two conventional bio-absorbable sutures 44. When the mesh 30 is implanted, the anchor-disc 42 remains positioned outside and against the vaginal skin of the subject's body to secure the vaginal skin in a hoisted position until the mesh is integrated with tissue and therefore secured in place. The bio-absorbable sutures 44 are selected such that they are absorbed into the subject's body after sufficient time for the mesh 30 to integrate with the tissue and become secured in place, at which time the anchor-disc 42 (which is attached to the mesh by the absorbable sutures) detaches from the mesh and is free to fall away from the subject's body.

In other embodiments, the distal and/or proximal anchors can be provided by other conventional anchor elements selected for providing the mesh-anchoring functionality described herein. For example, the distal and/or proximal anchors can be provided by a plurality of small distal and/or proximal barbs (e.g., of the type included on conventional barbed sutures) that extend from the mesh and can be hooked into the periurethral tissues to pull the mesh taught. In such embodiments, the barbs can be coupled to or formed integrally with the mesh such that they do not interfere with the emplacement of the mesh, but once the mesh is implanted in place, the barbs help secure the mesh to the adjacent tissue.

The mesh-tensioning assembly 36 of the surgical implant 12 is adapted for tensioning the implanted mesh 30 between the distal and proximal anchors 32 and 34. The tensioning assembly 36 can include at least one distal-tensioning string extending distally from the mesh 30 toward the distal anchor 32, slidingly engaging the distal anchor, and extending proximally toward the proximal anchor 34 so that the user can pull proximally on the tensioning string to thereby pull distally on the distal end 31 of the mesh to tension the mesh. As such, the engagement of the tensioning string and the distal anchor 32 functions to convert a proximal tensioning force applied to the tensioning string to a distal tensioning force applied to the mesh 30.

For example, the mesh-tensioning assembly 36 of the depicted embodiment includes a distal-tensioning string 46 and a lateral opening 48 in the distal anchor 32, with the tensioning string extending distally from the distal end 31 of the mesh 30, laterally through the distal-anchor opening, and proximally past the mesh and the proximal anchor 34. The tensioning string 46 can be provided by a conventional suture, string, cord, strip, or other flexible elongated element. The tensioning string 46 can be routed linearly alongside the mesh 30 or it can be woven through the mesh to aid in tensioning the mesh. Also, the tensioning string 46 can be routed through an opening (e.g., the depicted center axial aperture, or a notch, recess, or off-center opening) 49 in the proximal anchor 34 (e.g., in its disc-plate 42). The tensioning string 46 and the needle shaft 22 can extend through the same opening, or through separate/dedicated openings, in the proximal-anchor disc-plate 42. In this way, pulling on the free end of the tensioning string 46 below the proximal-anchor disc-plate 42 pulls the mesh 30 distally upward toward the distal anchor 32 to tension the mesh as desired. In addition, the opening 48 in the distal anchor 32 can be provided by an aperture (as depicted), notch, recess, or the like, that is formed in a tab 50 extending proximally from the distal anchor.

In this way, the tensioning string 46 and the distal-anchor opening 48 form a force-reversing pulley or winch mesh-tensioning assembly that converts the proximal/downward force on the tensioning string to the distal/upward force on the mesh 30 to tension the mesh. In alternative embodiments, other conventional types of force-reversing assemblies are implemented to convert a proximal/downward force on a tensioning string (or other tensioning element) into a distal/upward force on the mesh to tension the mesh. Such alternative force-reversing assemblies can include ratcheting systems, mini-gear systems, and the like. And in still other embodiments, instead of a force-reversing assembly that “pulls” on the distal end of the mesh to tension it, the tensioning assembly includes a “pushing” element that tensions the mesh by distally pushing on its distal end. Such a push-tension element can be integrally provided as a part of the implant or it can be separately provided (e.g., a conventional surgical tool that is received by a socket or catch at the mesh distal end).

Furthermore, the tensioning string 46 can include a series of unidirectional mechanical stops 52 with tapered leading surfaces and transverse trailing surfaces sized and shaped relative to the lateral opening 48 in the distal anchor 32 to provide for unidirectional advancement (see FIGS. 8-10). The mechanical stops 52 can be provided, for example, by wedge-shaped, Y-shaped, dome-shaped, or otherwise-tapered knots or other bodies formed integrally with or attached to the tensioning string 46. In some embodiments, the tensioning string 46 with unidirectional mechanical stops 52 is provided by a conventional barbed suture. The mechanical stops 52 can be pulled through the lateral opening 48 in the distal anchor 32 in one direction (as indicated by the right-side directional arrow of FIG. 10) by their tapered leading surfaces resiliently deflecting inward. But they cannot be pulled through the lateral opening 48 in the distal anchor 32 in the opposite direction (as indicated by the left-side directional arrow of FIG. 10), at least not without damaging the stops or opening, because their transverse trailing surfaces have a peripheral dimension larger than that of the opening to block the mechanical stops 52 from passing backward through the opening. In this way, once pulled through the opening 48, the stops 52 lock the tensioning string 46 in positions of incrementally increased tensioning with backward loosening prevented. This allows for postoperative adjustability by enabling the practitioner to tighten the tension on the mesh 30 for greater continence by pulling one or more additional mechanical stops 52 through the distal-anchor opening 48. For this reason, the tensioning string 46 is typically left in the vagina after the mesh 30 is implanted so that the practitioner can later (after initial emplacement) tighten the mesh, if needed.

In other embodiments, the tensioning string 46 is provided without the mechanical stops and secured (e.g., by tying or suturing) to itself, the mesh 30, the distal anchor 32, the proximal anchor 34, another part of the implant 12, or the subject's body to maintain the tension on the mesh. In some other embodiments, the mechanical stops 52 extend along a portion of the tensioning string 46 that is routed through the opening 49 in the proximal anchor 34 (instead of being located along the portion of the tensioning string is routed through the distal-anchor opening 48), and the proximal-anchor opening is sized and shaped relative to the mechanical stops to provide for the unidirectional advancement for tensioning.

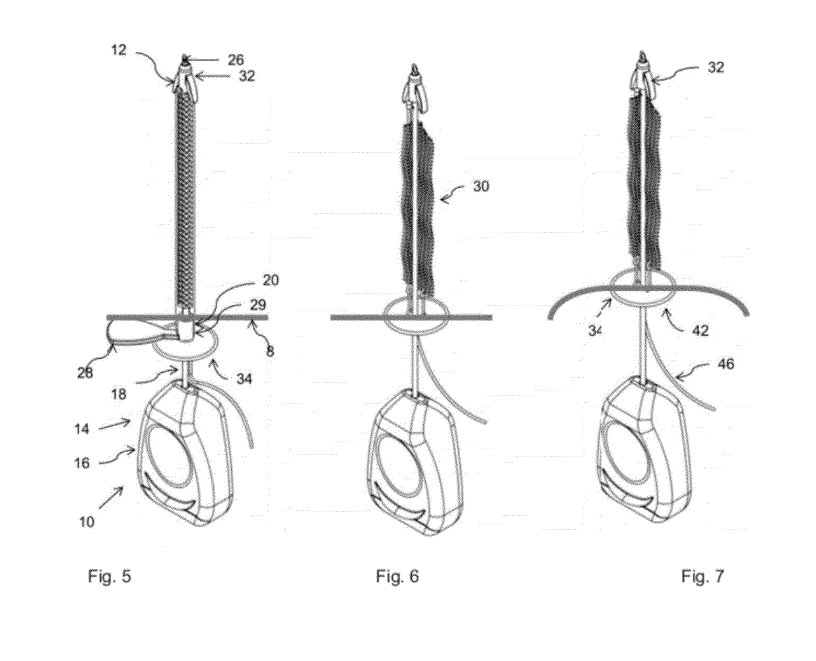

Referring primarily to FIGS. 5-14, a method of using the apparatus 14 to implant the implant 12 will now be described. FIG. 5 shows the surgical apparatus 14, holding the surgical implant 12, inserted into place within the subject's body. The practitioner so positions the system 10 by applying a distal-directed force to the handle 16, which causes the sharp distal end 26 of the needle 18 of the apparatus 14 to puncture the subject's skin 8 to enable the needle to be inserted into the subject's body into the desired position to emplace the mesh 30. The apparatus 14 is advanced until the distal anchor 32 is set in tissue in the desired position and the proximal anchor 34 is positioned outside and typically adjacent the skin 8. Then with the distal anchor 32 secured in place, the detachment member 28 is manipulated to pull the retainer 20 from the retaining closed position (FIG. 5) to the releasing opened position (not shown) and then off of the apparatus 14 (FIG. 6). This can be done by pulling on the detachment member 28 in a direction that is radially outward/lateral (to open the failure zone 29 and pull the retainer 20 from the retaining closed position to the releasing open position) and axially downward/proximal (to slide the released retainer off of the needle 18 and out of the subject's body). This in turn allows the resilient mesh 30 to unfurl and thus deploy from the stored position (FIG. 5) to the deployed position (FIG. 6).

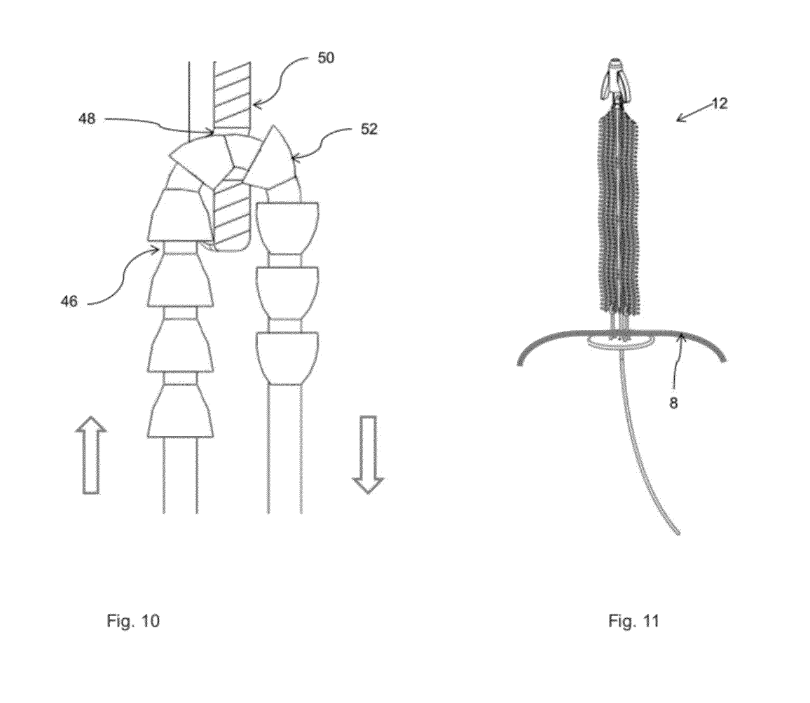

At this point, the practitioner pulls proximally on the tensioning string 46, which in turn pulls distally on the mesh 30 to pull it toward the distal anchor 32 and in turn to pull the proximal-anchor disc-plate 42 into its hoisted position (FIG. 7). Additionally or alternatively, the needle 18 can include a mechanical stop (e.g., a pin, tab, or other protrusion) above which the disc-plate 42 is positioned and held from proximal movement relative to the needle, and the practitioner can push distally on the handle 16 to hoist the disc-plate 42 and free up slack in the mesh 30 so that it can then by tensioned by pulling on the tensioning string 46. The unidirectional advancement and locking function of the mechanical stops 52 (FIGS. 8-10) locks the mesh 30 in this tensioned state. Then the apparatus 14 is removed, thereby withdrawing the needle 18 from engagement with the receiver 40 of the distal anchor 32 and from the subject's body, and thereby leaving the implant tensioned in place to treat the urinary incontinence or other medical condition (FIG. 11).

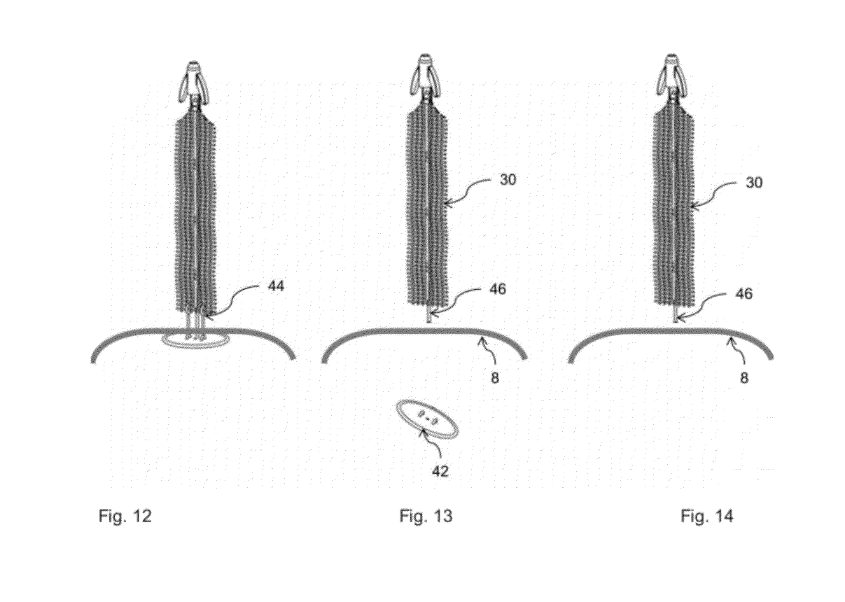

The implant 12 can be left in this position if the desired effects are attained, or if not then the practitioner can postoperatively further tension the mesh 30 by pulling the tensioning string 46 further to pull another one or more of the mechanical stops 52 through the distal anchor opening 48 (FIGS. 8-10). Once the effective tension of the mesh 30 has been achieved, the practitioner removes (e.g., cuts off) the excess portion of the tensioning string 46 extending out of the subject's body (FIG. 12).

Over time, the bio-absorbable connectors (e.g., sutures) 44 that connect the mesh 30 to the proximal anchor 34 are absorbed by the subject's body. When this happens, the proximal anchor 34 becomes detached from the proximal anchor 34 and is free to fall away (FIG. 13). The implant 12 is then left in this implanted position, providing the desired effective tension long-term, to treat the urinary incontinence or other medical condition (FIG. 14).

In other alternative embodiments, the distal end of the mesh is fixedly attached to the distal anchor, the tensioning string is not provided, and tensioning of the mesh is accomplished by pulling on a proximal-tensioning string extending proximally from the proximal end of the mesh. The proximal-tensioning string can have mechanical stops and the proximal anchor can be an opening through which the proximal-tensioning string extends, with the mechanical stops and the proximal-anchor opening cooperating to provide a unidirectional advancement for locking the proximal-tensioning string in place with the mesh tensioned.

Having described a first example embodiment of the invention, and numerous alternative embodiments thereof, additional example embodiments of the invention will now be described. It will be understood by persons having ordinary skill in the art that the features of any of the herein-described embodiments can be combined with features of other herein-described embodiments or of other non-disclosed embodiments to form additional embodiments of the invention not expressly disclosed herein.

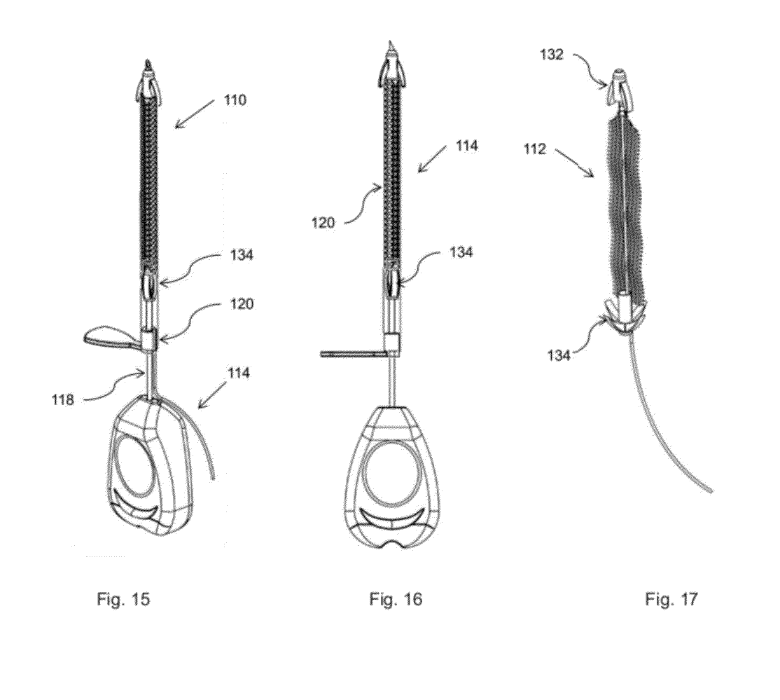

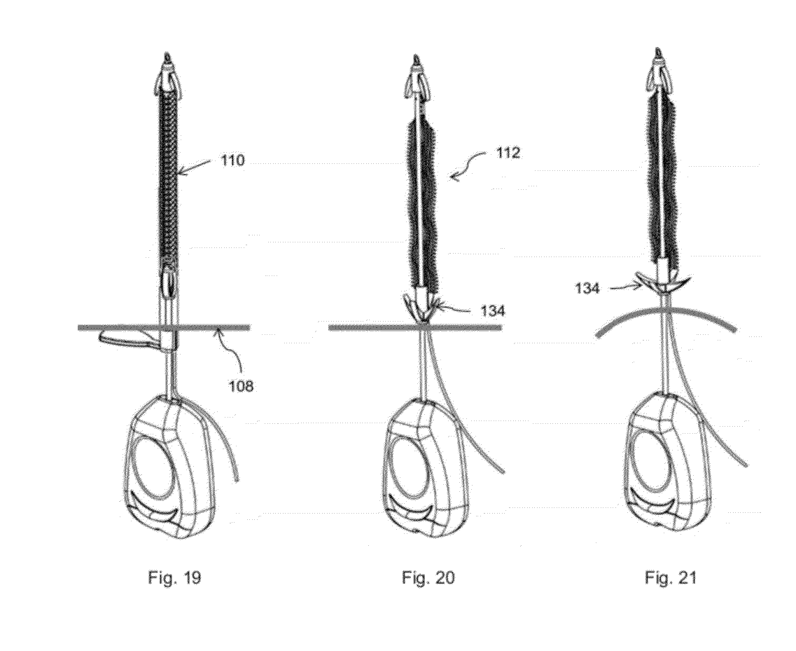

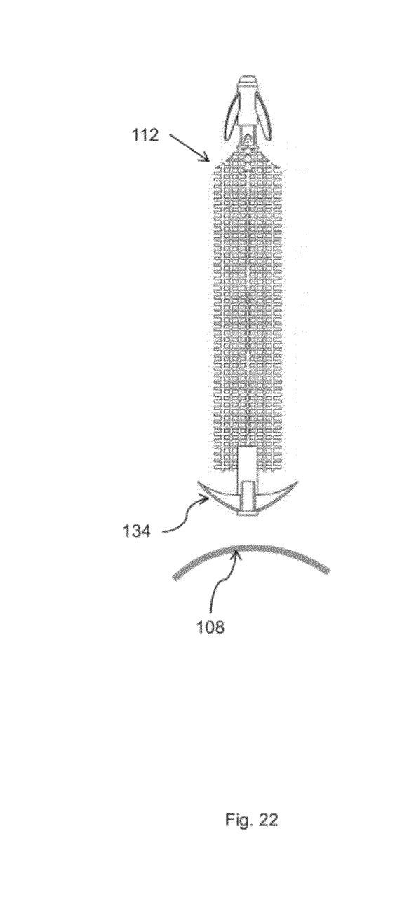

FIGS. 15-23 show a surgical system 110 according to a second example embodiment of the present invention, as well as a method of using the surgical system. The surgical system 110 (FIGS. 15-18) and method (FIGS. 19-22) are the same or similar to those of the first embodiment, with a few exceptions. As such, the surgical system 110 includes a surgical implant 112 and a surgical apparatus 114 for implanting the surgical implant through the subject's skin 108 and into their body. The surgical apparatus 114 can be identical to that of the first embodiment. And the surgical implant 112 can be identical to that of the first embodiment, except for the proximal anchor 134.

In this embodiment, instead of the exterior-positioned anchor-disc, the proximal anchor 134 includes a body 154 coupled to the mesh 130 and one or more foldable barbs 156 extending from the anchor body for positioning within the subject's body. The anchor body 154 can include an axial bore 158 through which the needle 118 extends and is retracted during use. Also, the anchor body 154 can be secured to the mesh 30 directly or by connectors such as bio-absorbable sutures. In some embodiments, the proximal anchor 134 (and/or the distal anchor 132) include one or more stops or notches to limit the bending of the barbs 156. And in some other embodiments, one or both of the distal and proximal anchors 132 and 134 are made of a bio-absorbable material that can be absorbed into the subject's body. Optionally, the barbs 156 can include one or more holes therethrough or indentations therein to help grip the tissue. And one or both of the distal and proximal anchors 132 and 134 can include a portion extending therefrom to which the retainer 120 is detachably coupled.

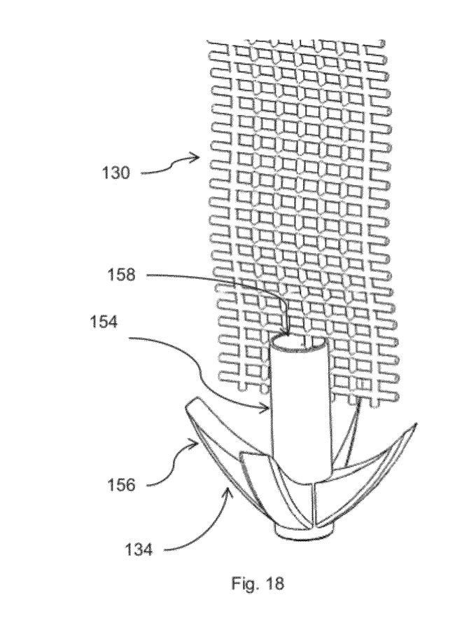

The foldable barbs 156 are resiliently biased radially outward, for example by including at least a portion made of a resilient material and/or including spring elements (e.g., bio-absorbable elastic distally-pulling sutures or distally-pushing coils) for such biasing. The barbs 156 are initially in a stored position in which they are resiliently deflected radially inward toward/against the anchor body 154 and held there by the retainer 120, for example within an outer tube retainer, in its retaining closed position (in which the retainer also holds the mesh 130 in the stored position) (FIGS. 15-16 and 19). But after the implant 114 has be inserted into place within the patient's body and the retainer 120 removed, the barbs 156 are free to resiliently deflect radially outward (FIG. 20). The barbs 156 then fully deploy to their radially outward extended positions in which they hook into tissue to secure the mesh 130 in place for tensioning (FIGS. 17-18 and 21-22).

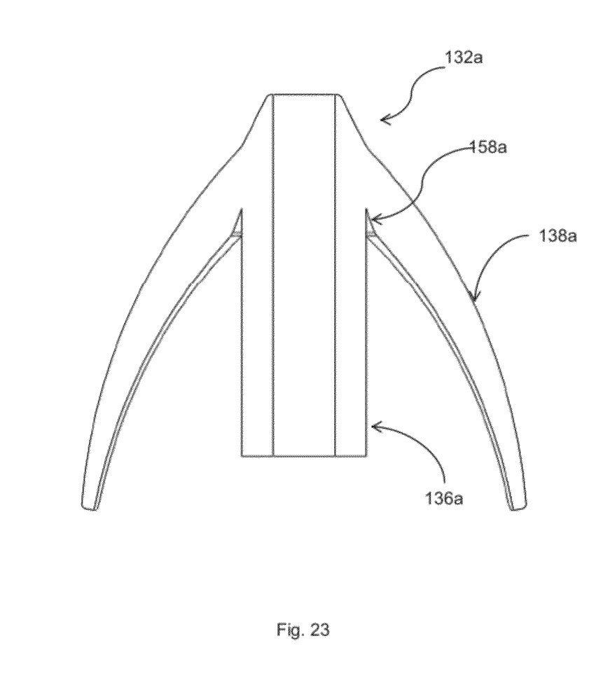

In addition, in an alternative embodiment the distal anchor 132a has a thinner/narrower body 136a and a recess (e.g., slit or notch on the acute-angle side) 158a where each of the barbs 138a extends from the anchor body (FIG. 23). This allows the barbs 138a to resiliently deflect inward to a stored position (not shown), for example held there by the retainer, where they are ready for deployment after the mesh is implanted and the retainer is removed.

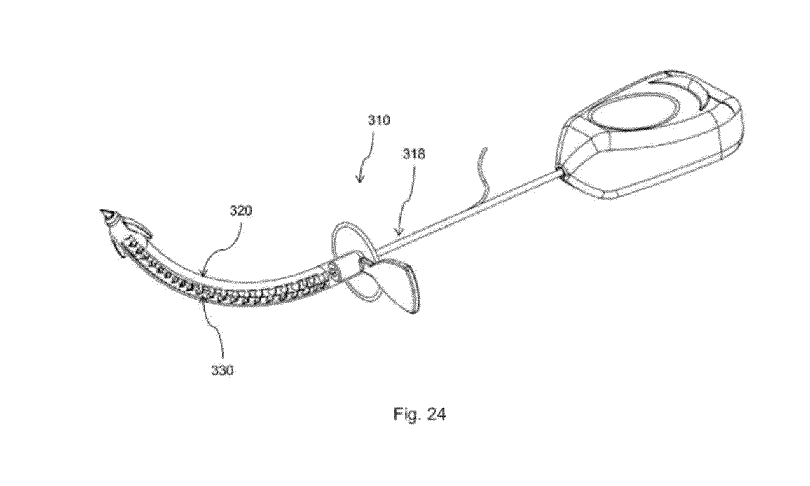

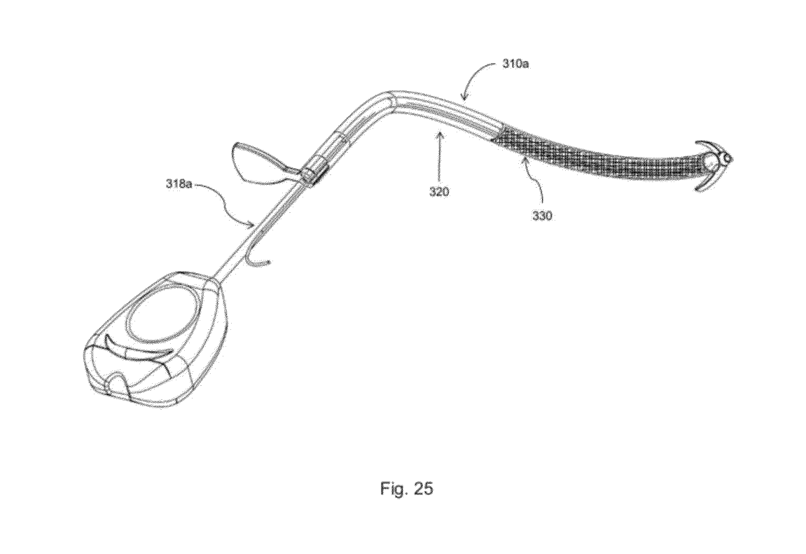

FIG. 24 shows a surgical system 310 according to a third example embodiment of the present invention, with major components that are the same as or similar to those of the first embodiment, with a few exceptions. In particular, the needle 318 of the surgical apparatus is not linear, as it is in the other embodiments depicted thus far. In the depicted embodiment, for example, the needle 318 is curved in one plane, and the flexible mesh 330 in the stored position and the flexible retainer 320 in the retaining closed position are in a conforming shape curved in one plane. In an alternative embodiment shown in FIG. 25, the surgical system 310a includes a needle 318a that is curved in two planes, and the flexible mesh 330a in the stored position and the flexible retainer 320a in the retaining closed position are in a conforming shape curved in two planes.

In some embodiments, the surgical mesh implant includes a laterally extending base at its proximal end for providing increased surface area for frictional securement in place. The laterally extending base moves from a stored inward position with a low profile to a deployed outward position in which it extends laterally to provide the additional surface area. The lateral base can be made of the same or a different biocompatible material as the vertical/longitudinal portion of the mesh.

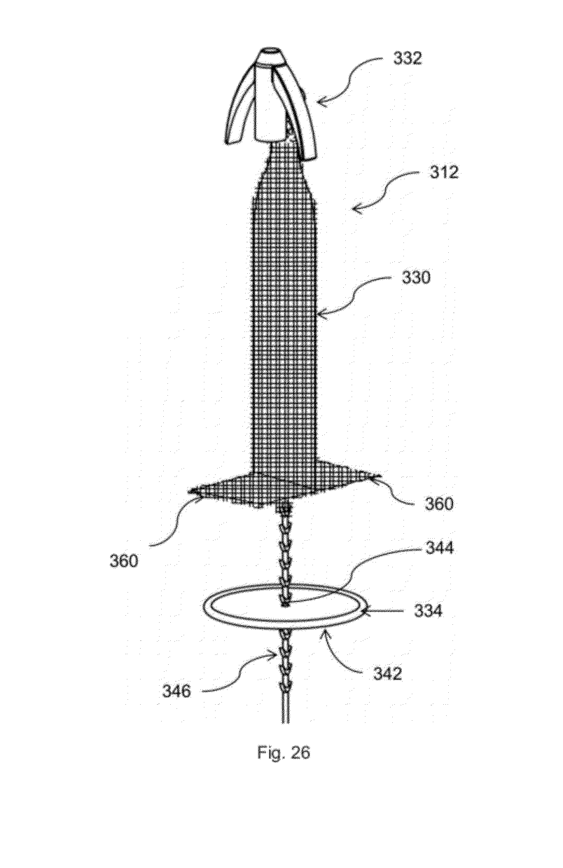

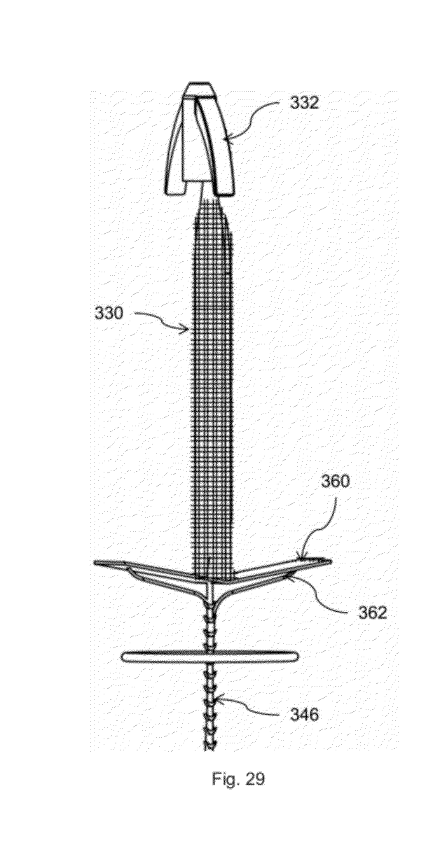

For example, FIGS. 26-30 show a surgical system 310 according to a fourth example embodiment of the present invention. The surgical system 310, and its method of use, are the same or similar to that of the first embodiment, with a few exceptions. As such, the surgical system 310 includes a surgical implant 312 and a surgical apparatus 314 for implanting the surgical implant through the subject's skin 308 and into their body. The surgical apparatus 314 can be identical to that of the first embodiment. And the surgical implant 312 can be identical to that of the first embodiment, except for the mesh 330.

In this embodiment, the surgical mesh 330 includes a laterally extending base in the form of two flat panels 360 extending laterally from opposite sides of the mesh at its proximal end 333 that cooperate with the mesh to take the general shape of an inverted “T” (in profile) when in the deployed position. The side panels 360 can be rectangular (as depicted) or they can have another regular or irregular shape (e.g., polygonal or semi-circular). In typical commercial embodiments, the mesh 330 has a length of about 25 mm and the bases 360 collectively have a length of about 16 mm, when the inverted “T” shape mesh is viewed in profile, and the width of the mesh/bases is about 8 mm, 6 mm, or 5 mm when the mesh is viewed from the top/distal end. In other embodiments, the side panels have curved edges and/or are deployed to a non-perpendicular position.

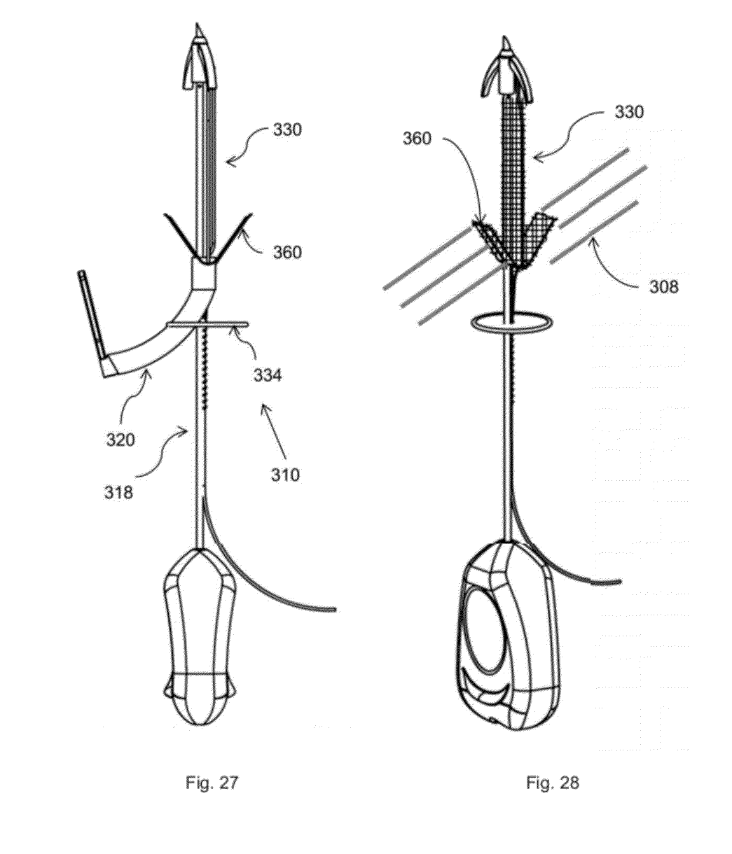

In order to deploy the side panels 360, they can include panel-deploying strings (e.g., sutures or cords) 362 extending between the free outer portions of the side panels and the tensioning string 346. In this way, after using the surgical system 310 to insert the needle 318 into the subject's body and remove the retainer tube 320 (FIGS. 27-28), the practitioner can pull on the tensioning string 346 to deploy the panels 360 down into the generally horizontal deployed position (while also distally tensioning the mesh 330).

In addition, the tensioning string 346 is routed up/distally from the mesh 330, through/around the distal anchor 332, and back down/proximally to past the mesh and proximal anchor 334, for tensioning the mesh (pulling downward/proximally on the tensioning string pulls the distal end of the mesh upward/distally, as described above). In this embodiment, the tensioning string 346 includes the mechanical stops 352 sized and shaped so that they provide unidirectional advancement through the opening 344 in the proximal anchor-disc 342 (the barbs and disc deflect/deform/compress slightly under pressure). But the one-way stops 352 retain the tensioning string 346 from sliding backwards relative to the anchor-disc 342 in the opposite direction. In this way, the practitioner can pull the tensioning string 346 to tension the mesh 330 as desired, and the mechanical stops 352 will then retain the string, and thus the mesh 330, in that tensioned position.

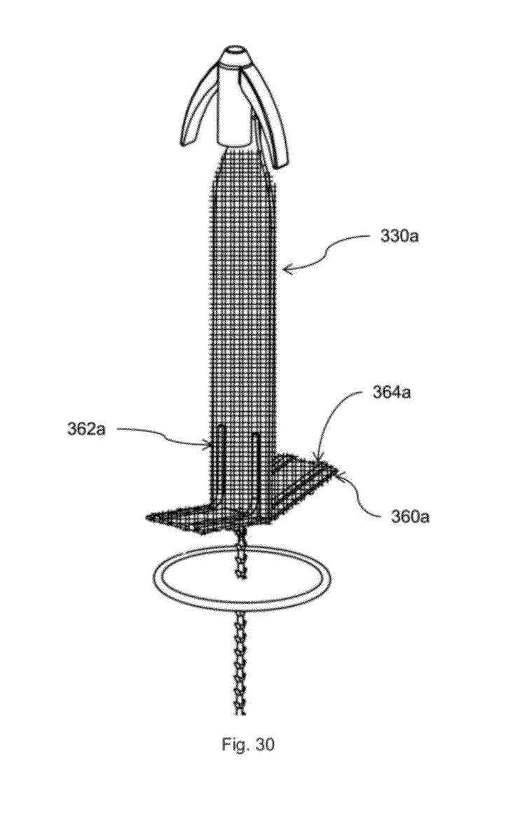

In an alternative embodiment shown in FIG. 30, the side panels 360a each include one or more elongated base-deployment members such as resilient plastic strips 364a attached to them and the vertical/longitudinal portion of the mesh 330a. The plastic strips 364a bias the side panels 360a to pivot outward into (or at least toward) a deployed outward position generally perpendicular to the vertical portion of the mesh 330a. The side panels 360a are retained in their stored inward position flush against the vertical mesh portion for example by the retainer tube (before its removal) and are free to deploy upon removal of the retainer tube.

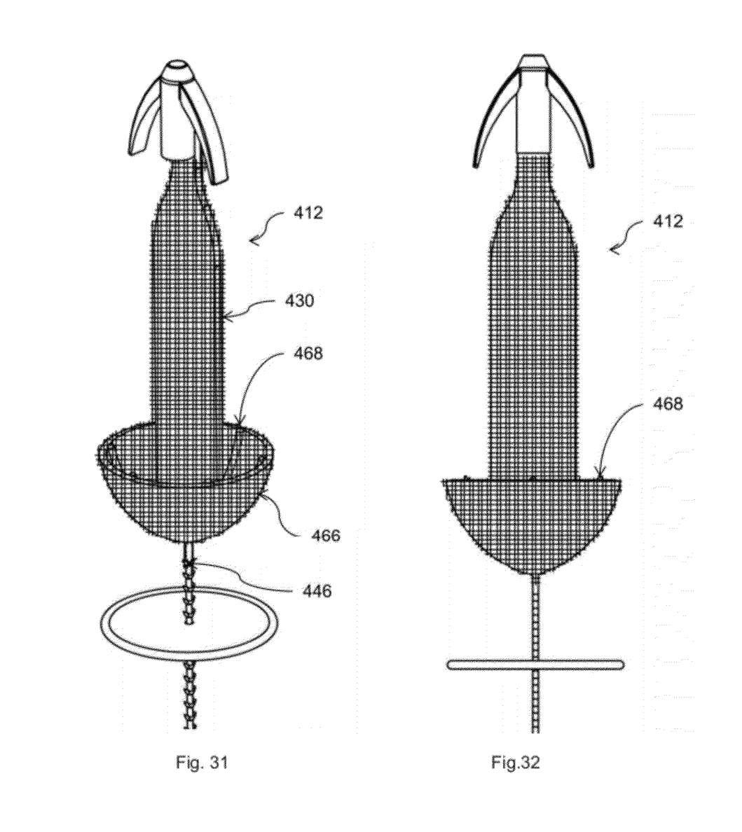

Similarly, FIGS. 31-32 show a surgical implant 412 of a surgical system according to a fifth example embodiment of the present invention. The surgical system, and its method of use, are the same or similar to that of the fourth embodiment, with a few exceptions. In particular, the surgical mesh 430 of the implant 412 of this embodiment includes a laterally extending base in the form of an upwardly extending dome (e.g., the bottom half of a generally spherical sheet) 466, with the tensioning string 446 extending generally centrally through it. When using the implant 412, the tissue layer “cups” against the outer surface/wall of the domed base 466 when it is extended outward to the deployed position from its inward stored position (not shown), with the curvature of the domed base providing increased surface area for frictional securement in place. The domed base 466 can include one or more elongated base-deployment members such as resilient plastic strings 468 for biasing the domed base outward into (or at least toward) the deployed outward position from the stored inward position flush against the vertical portion of the mesh 430. The domed base 466 is retained in its stored inward position flush against the vertical portion of the mesh 430 for example by the retainer tube (before its removal) and is free to deploy upon removal of the retainer tube.

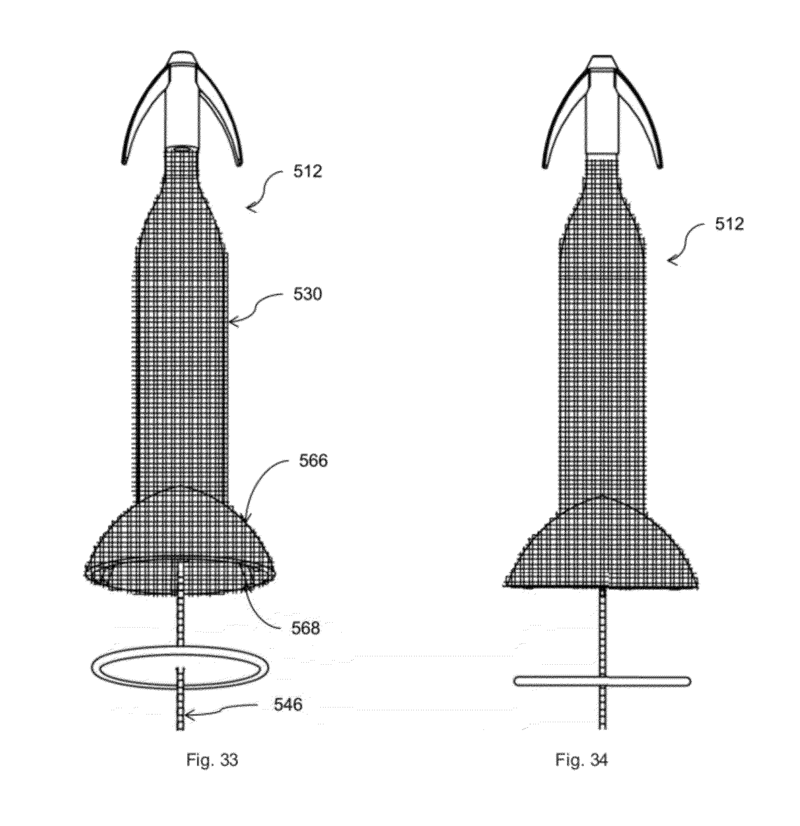

Also similarly, FIGS. 33-34 show a surgical implant 512 of a surgical system according to a sixth example embodiment of the present invention. The surgical system, and its method of use, are the same or similar to that of the fifth embodiment, with one exception. In particular, the surgical mesh 530 of the implant 512 of this embodiment includes a laterally extending base in the form of a downwardly extending dome (e.g., the top half of a generally spherical sheet) 566, with the tensioning string 546 extending generally centrally through it. When using the implant 512, the proximal anchor-disc 534 and tissue layer “cup” into the dome base for good securing when it is extended outward to the deployed position from its inward stored position (not shown), with the curvature of the domed sheet providing increased surface area for frictional securement in place. The domed base 566 can include one or more elongated base-deployment members such as resilient plastic strings 568 for biasing the domed base outward into (or at least toward) the deployed outward position from the stored inward position flush against the vertical portion of the mesh. The domed base 566 is retained in its stored inward position flush against the vertical portion of the mesh 530 for example by the retainer tube (before its removal) and is free to deploy upon removal of the retainer tube.

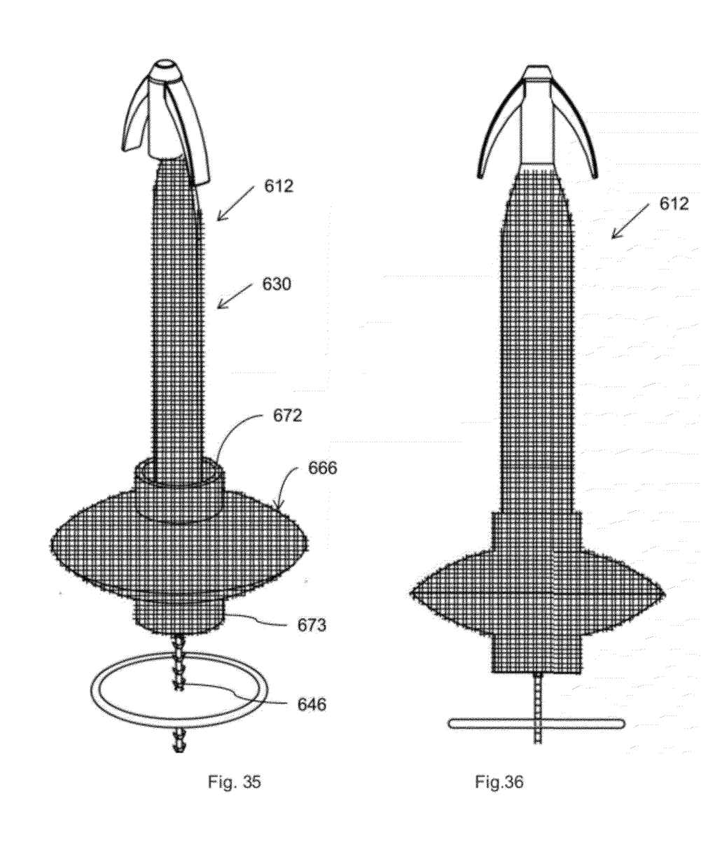

Also similarly, FIGS. 35-36 show a surgical implant 612 of a surgical system according to a seventh example embodiment of the present invention. The surgical system, and its method of use, are the same or similar to that of the fifth and sixth embodiments, with one exception. In particular, the surgical mesh 630 of the implant 612 of this embodiment includes a laterally extending base in the form of a collapsible/extendible 3D flexing saucer (e.g., an upper portion of a generally spherical sheet and a lower portion of a generally spherical sheet coupled or integrally formed together in an accordion-like, bellows-like, fashion) 666. A distal collar 672 can be fixedly attached between the distal end of the base 666 and the proximal end of the mesh 630. And a proximal collar 673 can be fixedly attached to the proximal end of the saucer base 666, with the tensioning string 646 extending generally centrally through it.

When using this embodiment, the proximal collar 673 can be pushed distally/upward (e.g., by a conventional surgical instrument) to compress the free bottom portion of the saucer base 666 against the attached top portion, thereby manipulating the base from its radially collapsed position (not shown) to its radially extended position (depicted). In the radially collapsed position, the saucer base 666 is longitudinally/axially elongated so that it is flush relative to the mesh 630 (it does not extend laterally outward beyond, or much beyond, the mesh). And in the radially extended position, the saucer base 666 is longitudinally/axially compressed so that its middle portion is forced radially outward to provide increased surface area for frictional securement in place. The saucer base 666 can be retained in its stored inward position flush against the vertical portion of the mesh 630 for example by the retainer tube (before its removal) and can be free to deploy upon removal of the retainer tube. In other embodiments, the saucer base includes one or more elongated base-deployment members such as resilient plastic strings for biasing the saucer base into (or at least toward) the deployed outward position from the stored inward position.

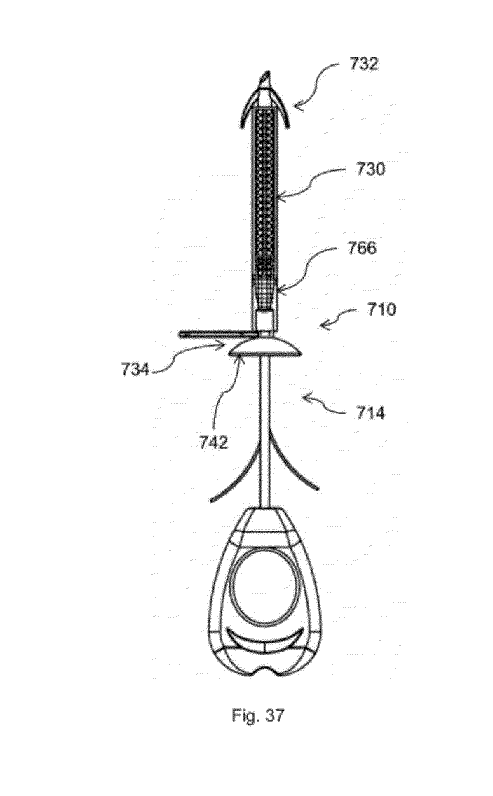

And also similarly, FIGS. 37-48 show a surgical system 710, and its method of use, according to an eighth example embodiment of the present invention. The surgical system 710, and its method of use, are the same or similar to that of the fifth through seventh embodiments, with exceptions. In particular, the surgical mesh 730 of the implant 712 of this embodiment includes a laterally extending base in the form of a parachute dome (e.g., the top portion of a generally spherical sheet, similar to the domed base 566 described above) 766 when deployed. And the proximal anchor 734 is in the form of an anchor-disc 742 with a generally conforming convex domed shape, so that the two mate together with the tissue layer between them to further promote the application of the mesh base 766 to the inner surface of the vagina along each fornix.

Referring primarily to FIGS. 37-40, one or more (e.g., one as depicted) distally-tensioning strings 746 extend upward/distally from the distal end of the mesh 730, laterally through/around the distal anchor 732, back down along the length of the mesh 730 and the parachute-domed base 766 (e.g., generally centrally therethrough), and generally centrally through the proximal anchor-disc 742, similarly to that described above. And a plurality of base-deploying strings 768 extend from the free peripheral edge(s) of the parachute-domed base 766, can be routed through the opening 749 in the proximal anchor-disc 742, and can converge together into converged/main base-deploying string 769 (the domed base and the plural base-deploying strings together having the general appearance of a parachute). In some embodiments, the base-deploying strings 768 extend along the length of the parachute-domed base 774, and in some such embodiments these portions of the base-deploying strings are provided by resilient strips that bias the parachute-domed base from its stored inward position to its deployed outward position.

The mesh-tensioning string 746 and the main base-deploying string 769 (or the individual base-deploying strings 768) can be coupled together below/proximal to the proximal anchor-disc 742 by a crimp, knot, etc. so that they can be manipulated together and to provide a mechanical stop (too large to fit back up distally through the anchor-disc opening 749) for retaining the mesh 730, the parachute-domed base 766, and the anchor-disc in place. In addition, in this way the coupled-together mesh-tensioning string 746 and main base-deploying string 769 (or the individual base-deploying strings 768) can be pulled together to further tension the mesh 330 and the parachute-domed base 766 into a desired position and state.

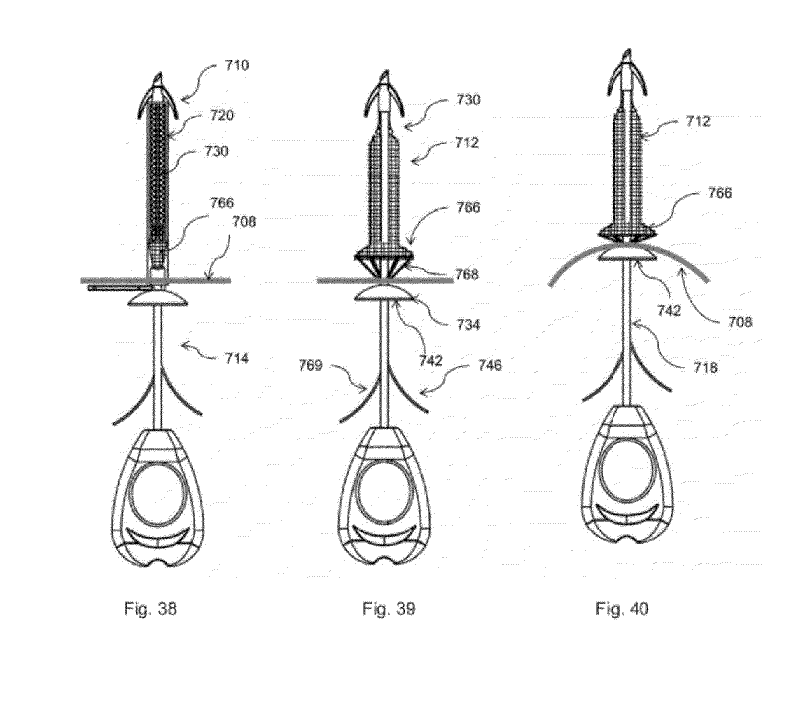

In use, the apparatus 714 is used to insert the mesh 730 through the subject's skin 708 and into their body, the retainer 720 is removed, and the mesh 730 is deployed (FIGS. 38-39). The parachute-domed base 766 deploys from its stored inward/downward position to its deployed outward/downward position when the retainer tube 720 is removed because the base is made of a resiliently deformable material and/or includes resilient tensioning members (e.g., plastic strips or strings). The curvature of the parachute-domed base 766 in the deployed position provides increased surface area for frictional securement in place.

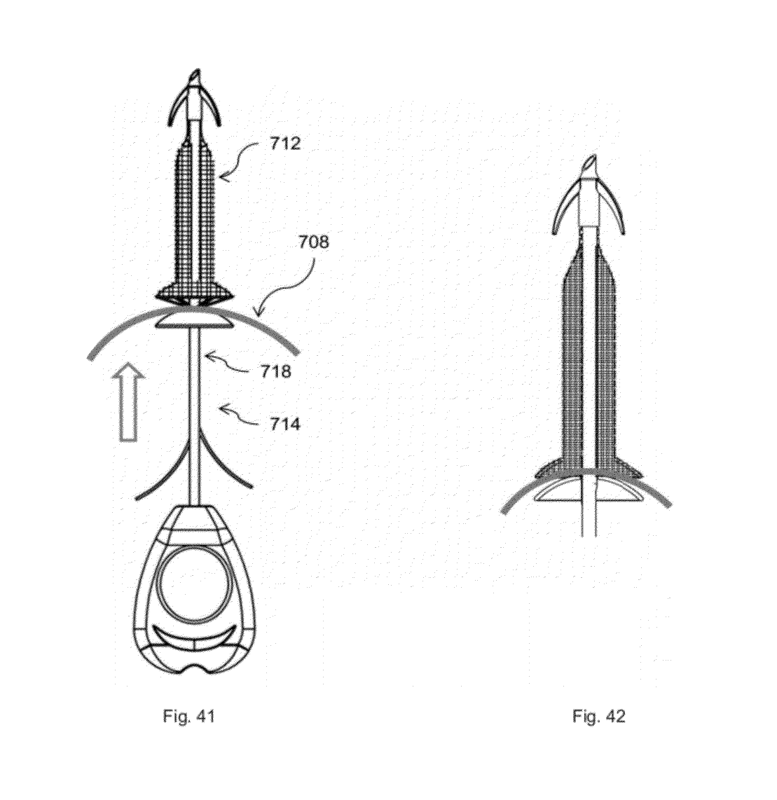

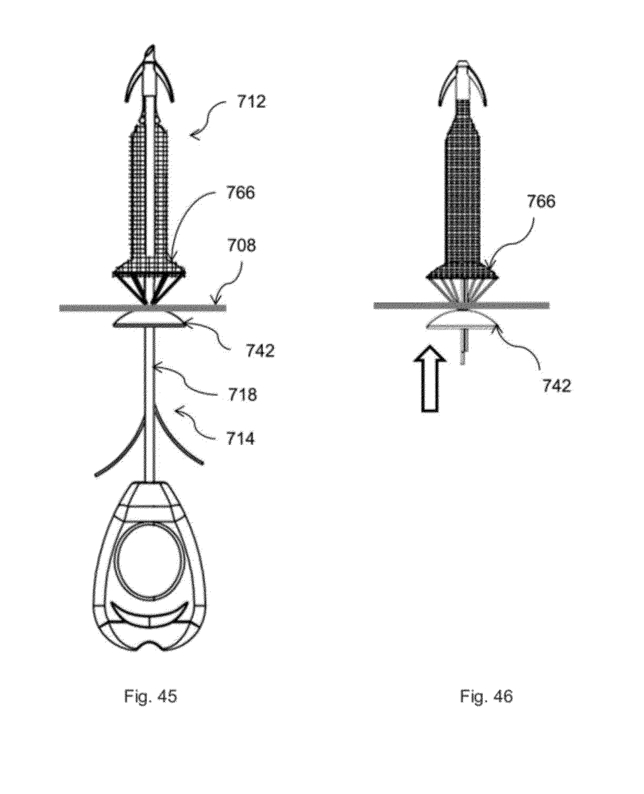

Then the needle 718 is advanced distally to advance the convex anchor-disc 742 into proximity to the base 766, with the skin 708 between them, to cause the flexible base to conform to the shape of the less-flexible convex anchor-disc (FIG. 40). The parachute-domed base 766 is thereby further expanded/flattened outward to conform to the shape of the convex anchor-disc 742 as the convex anchor-disc is forced upward against it (with the skin 708 therebetween) while maintaining tension on the base-deploying string(s) 769(768). In a first optional method (FIGS. 41-44) this is done by advancing the needle 718 upward/distally and in a second option (FIGS. 45-48) this is done without the needle by advancing the convex anchor-disc 742 upward/distally.

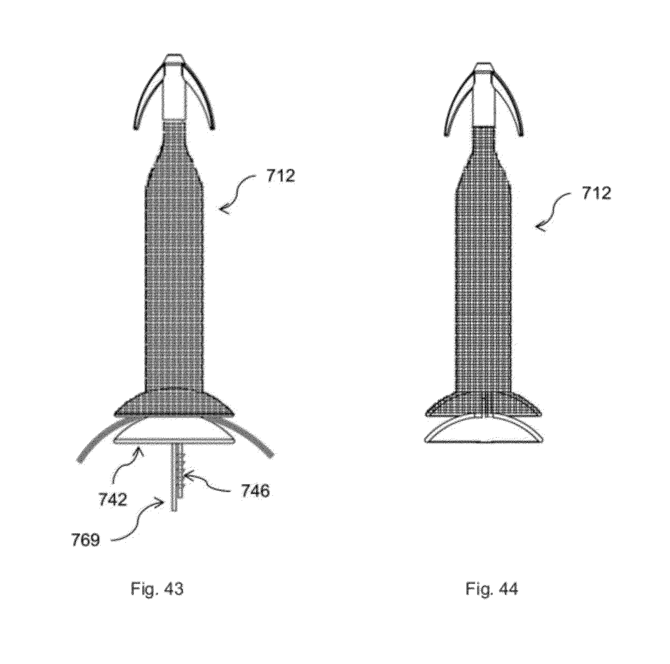

Referring to the first option, in FIG. 41 the implant 712 is emplaced and deployed by the apparatus 714, with the needle 718 advanced to conform the parachute-domed base 766 to the convex anchor-disc 742 while maintaining tension on the main/converged base-deploying string 769. In FIG. 42 the needle 718 is removed, in FIG. 43 the mesh-tensioning string 746 and the converged base-deploying string 769 are coupled together distally of the proximal anchor-disc 742, and in FIG. 44 the coupled-together mesh-tensioning string and converged base-deploying string are removed to leave the finished implant in place.

And referring to the second option, in FIG. 45 the implant 712 is emplaced and deployed by the apparatus 714, and in FIG. 46 the needle 718 is removed, with the convex anchor-disc 742 advanced to conform the parachute-domed base 766 to the convex anchor-disc (without using the needle 718) while maintaining tension on the main/converged base-deploying string 769. In FIG. 47 the mesh-tensioning string 746 and the converged base-deploying string 769 are coupled together distally of the proximal anchor-disc 742, and in FIG. 48 the coupled-together mesh-tensioning string and converged base-deploying string are removed to leave the finished implant in place.

In an alternative embodiment, the parachute-domed base in the stored position is in an inward/upward position (in the manner of the upward-dome embodiment of FIGS. 31-32) instead of an inward/downward position (in the manner of the downward-dome embodiments of FIGS. 33-34 and 37-48). In such embodiments, pulling on the base-deploying string deploys the parachute-domed base from the stored inward/upward position distally down into the deployed outward/downward position, with the base made of a sufficiently rigid material that this does not cause the base to collapse radially inward. In another alternative embodiment, the parachute-domed base deploys because of a combination of being made of a resilient material, including resilient tensioning members, and/or being tensionable by base-deploying strings.

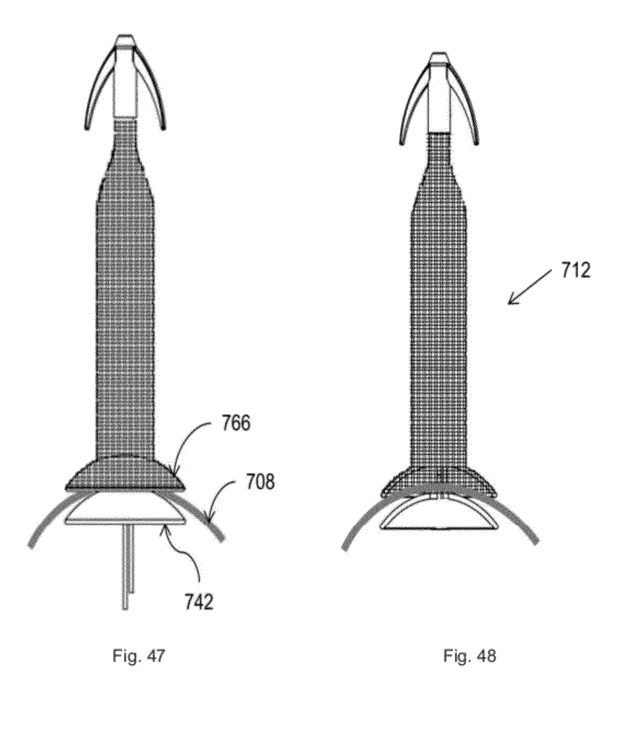

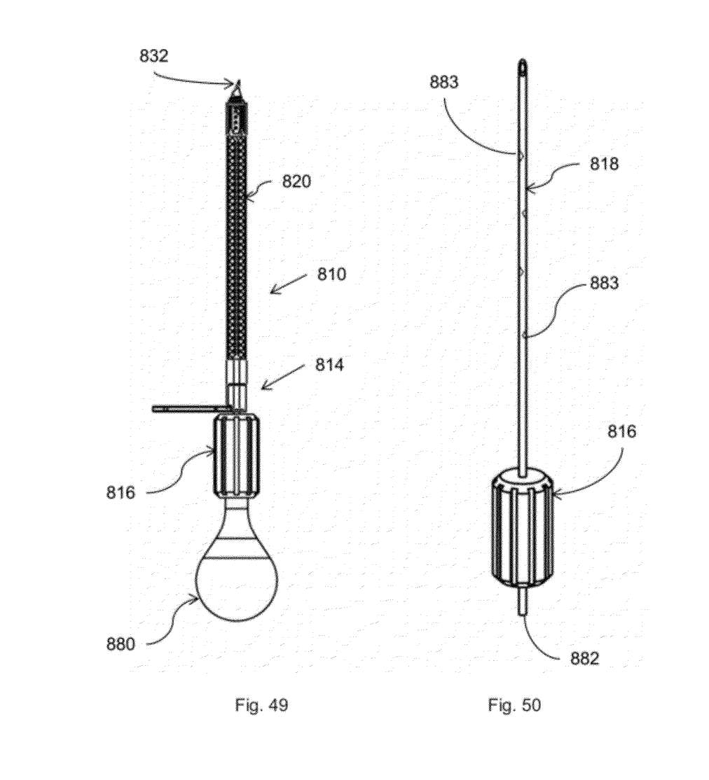

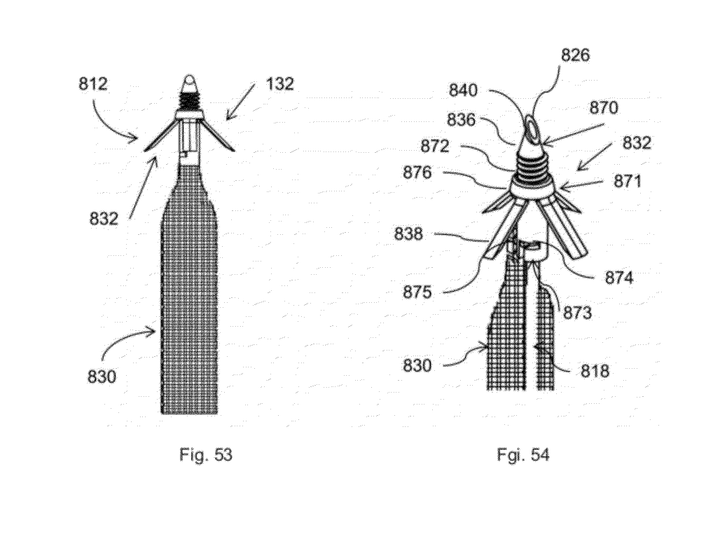

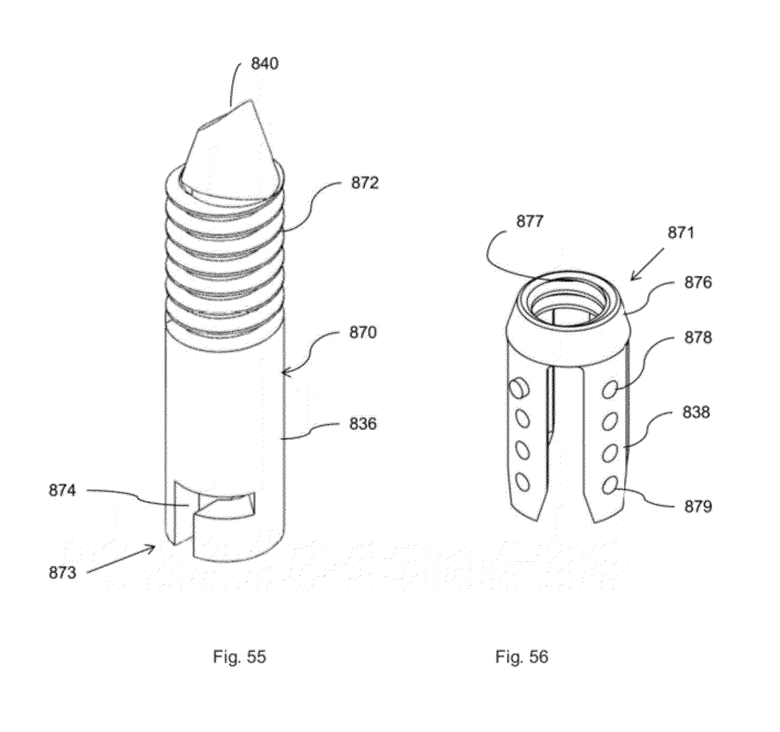

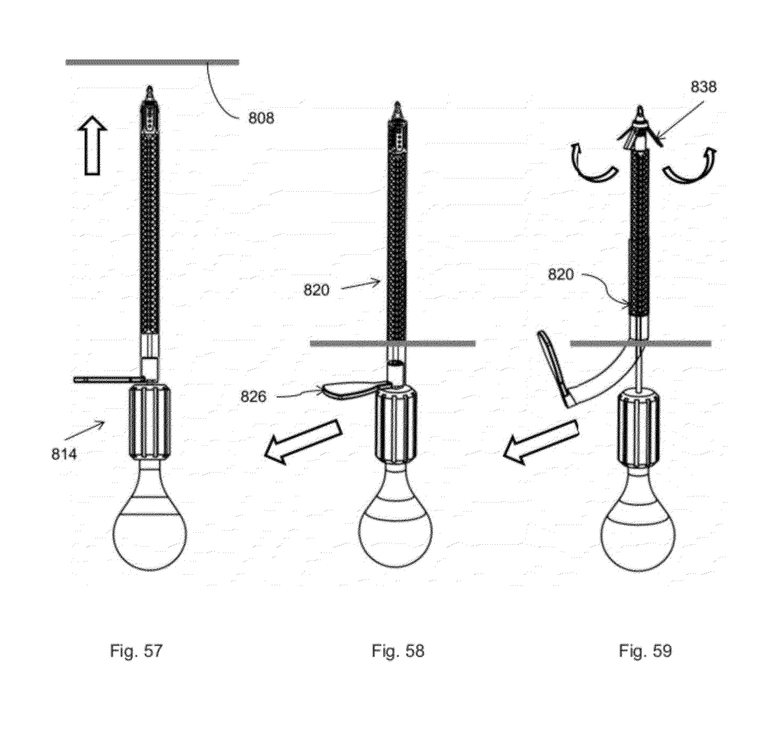

FIGS. 49-67 show a surgical system 810 according to a ninth example embodiment of the present invention, as well as a method of using the surgical system. The surgical system 810 (FIGS. 49-56) and method (FIGS. 57-67) are the same or similar to those of the other herein-described embodiments, with a few exceptions. As such, the surgical system 810 includes a surgical implant 812 and a surgical apparatus 814 for implanting the surgical implant through the subject's skin 808 and into their body. The apparatus 814 includes a handle 816 (e.g., cylindrically shaped), a needle 818 extending therefrom, and a retainer 820 (e.g., a tube with a perforated failure zone 829 and a detachment member 826) that is fitted onto the needle. The implant 814 has a mesh 830, a distal anchor 832 (which has a different form in this embodiment), a mesh-tensioning assembly 836 (which has a different form in this embodiment), and a non-integral proximal anchor 834 (which has a different form in this embodiment).

In this embodiment, the mesh-tensioning assembly is adapted to distally advance the distal anchor 832 to tension the mesh 830 by the application of a rotary force. In the depicted embodiment (FIGS. 54-56), for example, the distal anchor 832 includes a rotary element 870 and a securing element 871 that is anchored in place by rotation of the rotary element, which in turn distally advances the rotary element to tension the mesh 830. The rotary element 870 includes the anchor body 836 with the needle receiver 840 (e.g., a central bore through which the needle tip 826 coaxially extends and fits so that the anchor body rotates with the needle 818 in a first rotary direction), external threading 872, and at least one element of a releasable coupling assembly 873. The mesh 830 (e.g., its distal end) is attached to the anchor body 836.

The releasable coupling assembly 873 can be provided by a rotary-release bayonet assembly including an “L” slot 874 in the distal-anchor body 836 and a pin 875 extending from the needle 818. These components can be configured as shown so that upon rotation of the needle 818 in one direction the force of the pin 875 against the endwall of the lateral portion of the L-slot 874 causes the distal-anchor body 836 to co-rotate therewith. But rotation of the needle 818 in the opposite direction (with the securing element 871 frictionally engaged by tissue and thereby influenced against co-rotating therewith) causes the pin 875 to travel along the lateral portion of the L-slot 874 until the pin aligns with the axial portion of the L-slot (position shown in FIG. 54). In that position, the needle 818 can be proximally retracted and thereby withdrawn from operable connection to the distal anchor 832, leaving the distal anchor secured in place.

And the securing element 871 includes a collar 876 with the barbs 838 extending therefrom, internal threading 877 that mates with the external threading of the rotary element 870 of the anchor body 836, and the pin 875 (or another element) of the releasable coupling assembly 873. In addition, the barbs 383 can include at least one protrusion (e.g., a pin, boss, ramp, or other projecting element) that engages a cooperating element (not shown) on the retainer tube 820 for holding the retainer in place during needle insertion. Also, the barbs 383 can include a series or array of openings (e.g., holes, recesses, notches, or other openings) that receivingly engage the tissue to help secure the distal anchor 832 in place.

The needle 818 rotates with the handle 816, for example, they can be attached together by a friction/interference fit. Accordingly, the handle 816 can be rotated and pushed distally to rotate the needle 818 and thus the body 836 of the distal-anchor rotary element 870. But with the barbs 838 deployed and engaging tissue, the distal-anchor securing element 871 does not rotate with the rotary element 870. Instead, the mating threads 872 and 877 cause the rotary element 870—to which the mesh 830 is attached—to advance distally (while the barbs 838 do not advance distally), thereby tensioning the mesh.

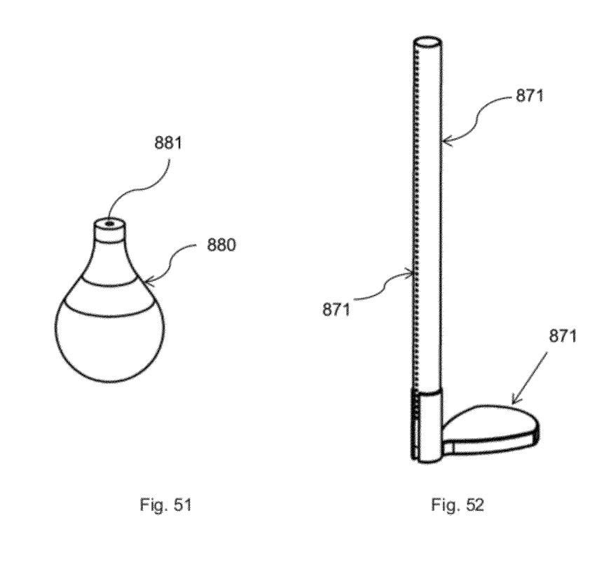

In addition, the proximal anchor 834 of this embodiment is different from what is described elsewhere herein. Instead of a barbed anchor, the proximal end of the mesh 830 is secured in place by epoxy anchors, and the apparatus 814 and implant 812 include modifications to permit this. In the depicted embodiment, for example, the apparatus 814 includes a glue dispenser 880 with an outlet opening 881 in fluid communication with a lumen 882 extending axially all the way through the needle 818 (from its proximal to its distal end), and with the needle including radial/lateral glue ports 883 through which glue can delivered from the needle lumen onto the mesh 830 (FIGS. 50-51). The glue dispenser 880 can be provided by a bulb/pump that defines an internal reservoir for holding one or more fluids such as epoxy (in the case of the present embodiment) that can be forced (e.g., by squeezing the dispenser bulb) into and through the needle lumen 882 and delivered to a surgical site distal of the dispenser. The glue dispenser 880 can be attached to the handle 816 (directly or indirectly via the needle 818) so that they rotate together, or they can be rotationally independent (e.g., by an interposed rotational bearing).

The needle 818 can be formed with a closed distal end 823, or an open end that is occluded, to prevent the glue from being discharged therefrom. Alternatively, the needle can have an open distal end and a fluid (e.g., glue and/or an anesthetic) can be dispensed out through it. Alternatively or additionally, the same or a different fluid dispenser containing another fluid such as an anesthetic can be coupled to the needle such that the anesthetic can be delivered through the needle lumen and to a surgical site.

In typical commercial embodiments, the glue dispenser 880 is about 15 mm long, the handle 816 is about 15 mm long, the needle 818 is about 60 mm long, the mesh 830 is about 30 mm long, and the distal anchor 832 is about 10 mm long. Also, the outer diameter of the needle 818 is about 2.2 mm, that of the distal anchor collar 876 (with the barbs 838 in their radially inward stored position) is about 60 mm, and that of the retainer tube 820 is about 3.6 mm. And the width of the mesh is about 6 mm. Of course, the system 810 can be provided in other dimensions as may be desired.

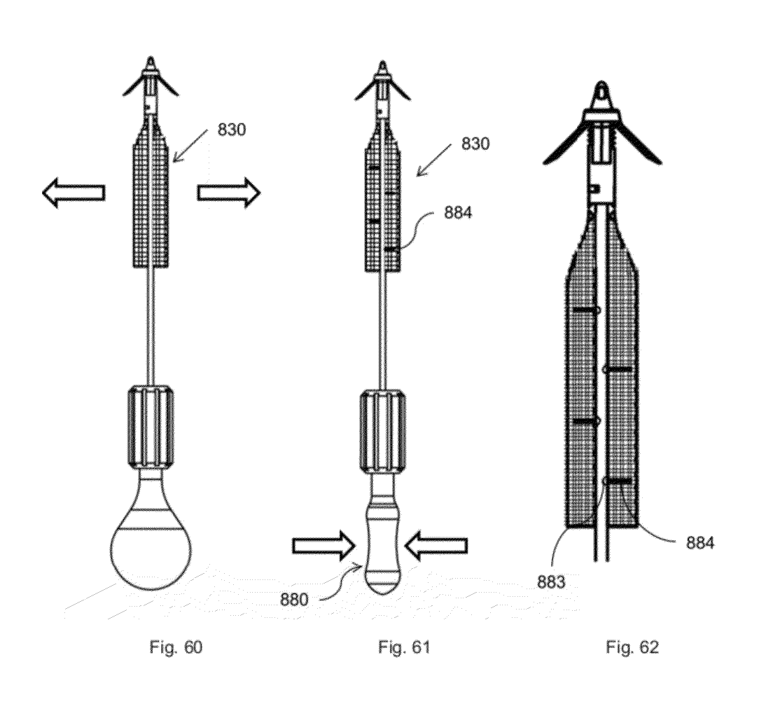

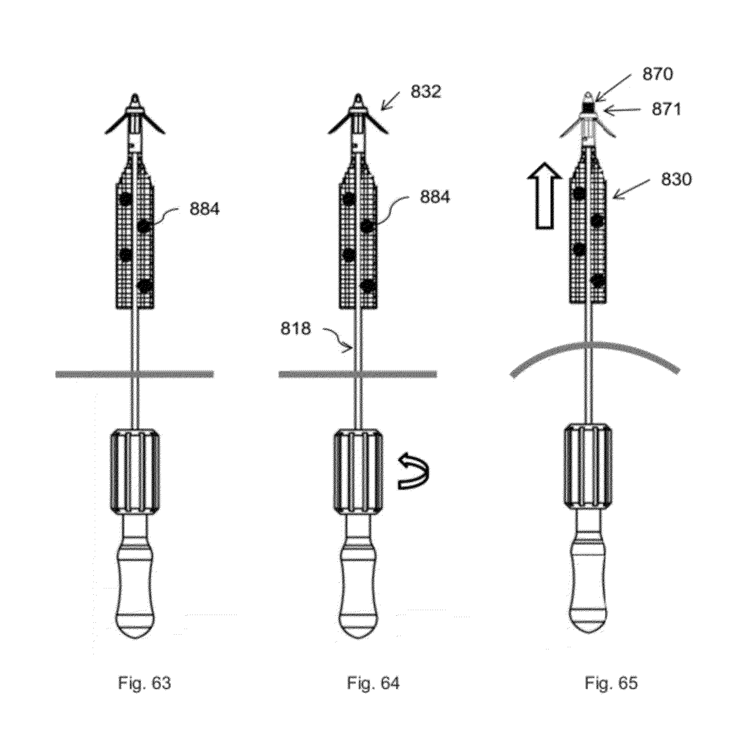

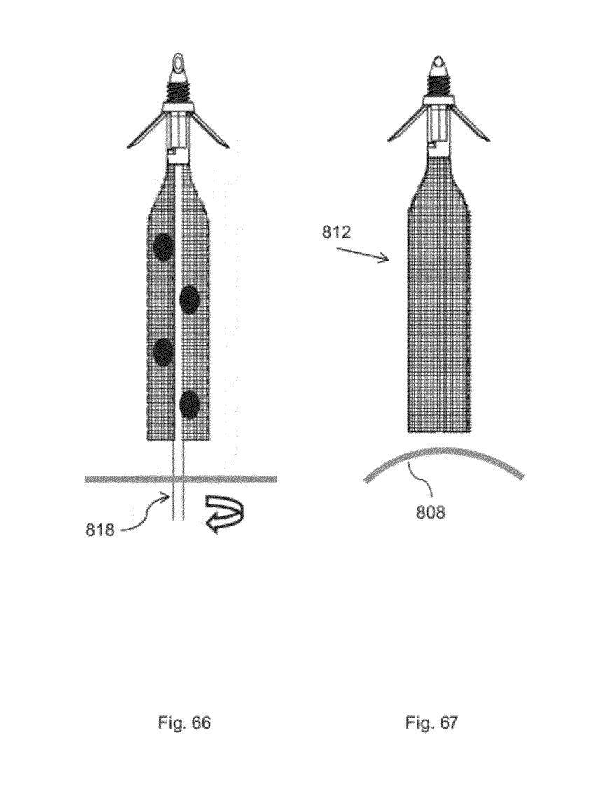

Having described different structural aspects of this embodiment, details of its use will now be described. The apparatus 814 is used to insert the needle 818 into place, the retainer 820 is removed, and the released distal-anchor barbs 838 and mesh 830 spring out laterally into their deployed positions (FIGS. 57-60). Then the dispenser 880 is actuated to deliver the glue through the needle's glue ports 883 and onto the mesh 830 where they form glues spots 884 (FIGS. 61-62). The glue spots 884 are allowed to dry or otherwise cure until they bind the mesh 830 to tissue (FIG. 63). Then the needle 818 is rotated and distally advanced to in turn rotate and distally advance the rotary element 870 of the distal anchor 832, while the barbs 838 of the securing element 871 of the distal anchor remain in place, and while the glue spots 884 secure those locations of the mesh 830 in pace, to thereby distally advance the distal end of the mesh to tension the mesh (FIGS. 64-65). Finally, the needle 818 is reverse-rotated to release it from the distal anchor 832 and proximally retracted to withdraw it from the subject's body, and the implant 812 is left tensioned in place in a sling-like fashion (FIGS. 66-67).

The locations of the glue ports 883 on the needle 818 are selected to position the glue spots 884 where desired on the mesh 830. As depicted, the glue spots 884 are positioned along the length of the mesh 830. If desired, they can be concentrated near the proximal end of the mesh 830 to enable adding tension along more of the length of the mesh.

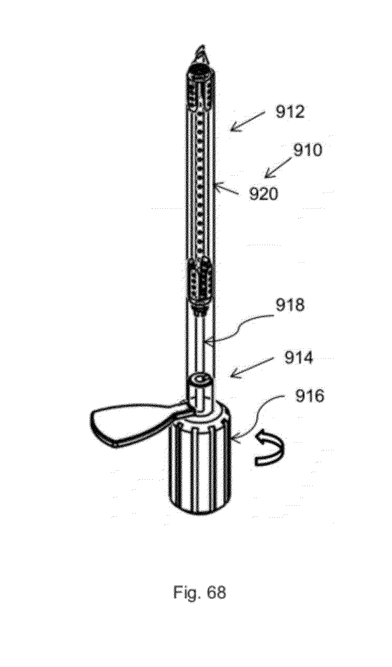

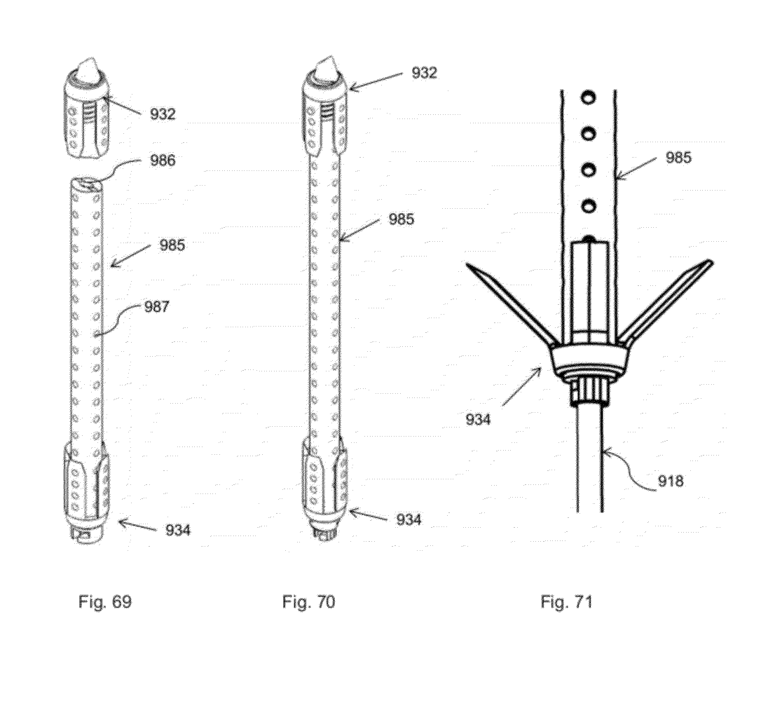

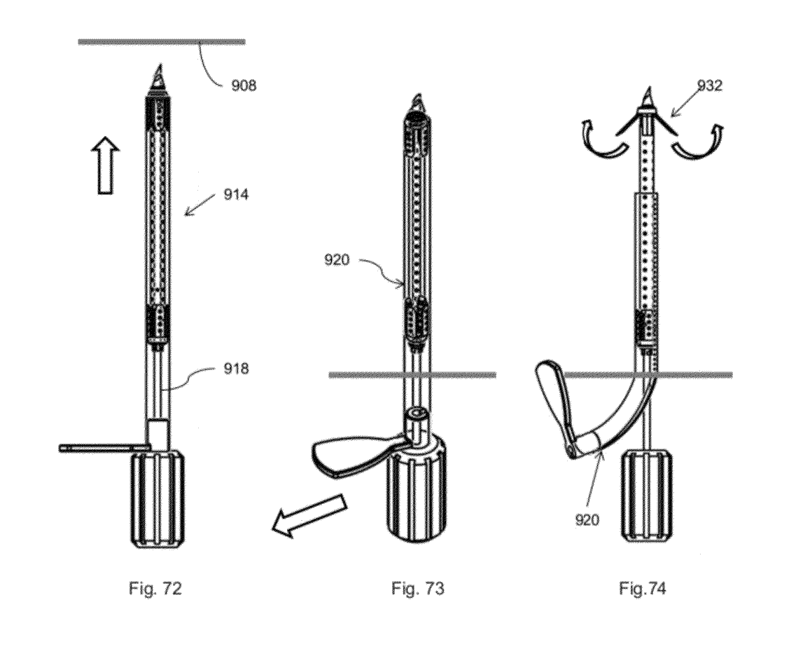

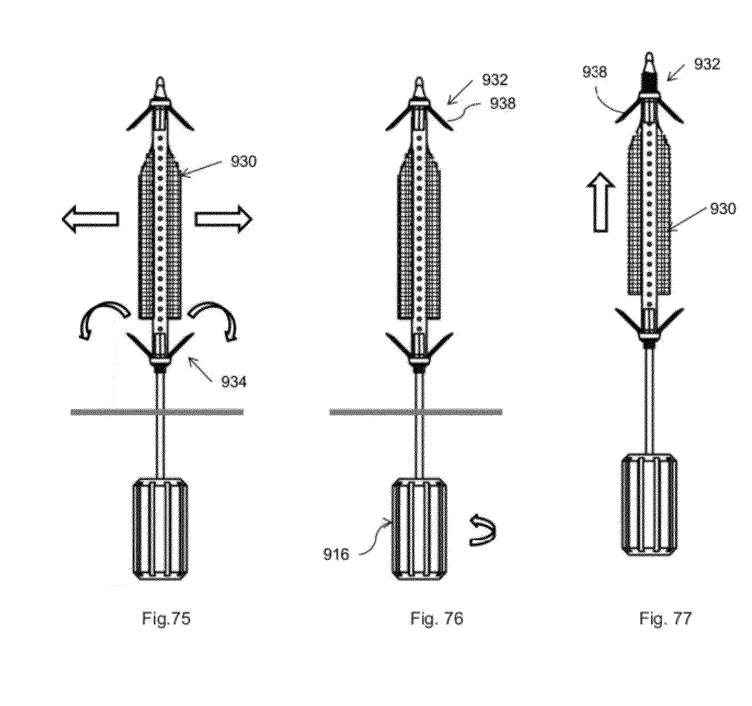

FIGS. 68-80 show a surgical system 910 according to a tenth example embodiment of the present invention, as well as a method of using the surgical system. The surgical system 910 (FIGS. 68-71) and method (FIGS. 72-80) are the same or similar to those of the other herein-described embodiments, with a few exceptions. As such, the surgical system 910 includes a surgical implant 912 and a surgical apparatus 914 for implanting the surgical implant through the subject's skin 908 and into their body. The apparatus 914 includes a handle 916, a needle 918 extending therefrom, and a retainer 920 fitted onto the needle. The implant 914 includes a mesh 930, a distal anchor 932 and mesh-tensioning assembly (e.g., a rotationally adjustable mesh-tensioning anchor as in the immediately preceding embodiment), and a proximal anchor 934.

In this embodiment, the implant 912 additionally includes a connector shaft 985 extending between and attached to the distal anchor 932 and the proximal anchor 934. The connector shaft 985 includes a lumen 986 through which the needle 918 is received and held during implantation. In some embodiments such as that depicted, the connector shaft 985 also includes a plurality of lateral ports 987 through which a fluid is delivered. For example, the ports 985 can be used to deliver a medication (e.g., anesthesia) to the surgical site by inserting a syringe into the lumen 986 of the connector shaft 985 after the needle 918 is removed. Or the ports 985 can be used to deliver a glue to the mesh 930 to form distal or proximal anchors securing (or at least contributing to the securement) of the mesh in place.

The connector shaft 985 is made of a bio-absorbable material that is absorbed into the body over time. And in embodiments such as that depicted, in particular when using epoxy to provide additional proximal anchoring, the distal anchor 934 is also made of a bio-absorbable material that is absorbed into the body over time. Thus, the connector shaft 985 and the distal anchor 934 are implanted during the surgical procedure so provide stability and securement for the mesh 930 until the glue forms a stronger bond to the tissue, and then they are absorbed into the body so a separate surgical procedure is not needed to remove them. The lateral ports 987 provide the additional benefit of there being less material to be absorbed by the body, without sacrificing the structural benefit of the connector shaft 985.

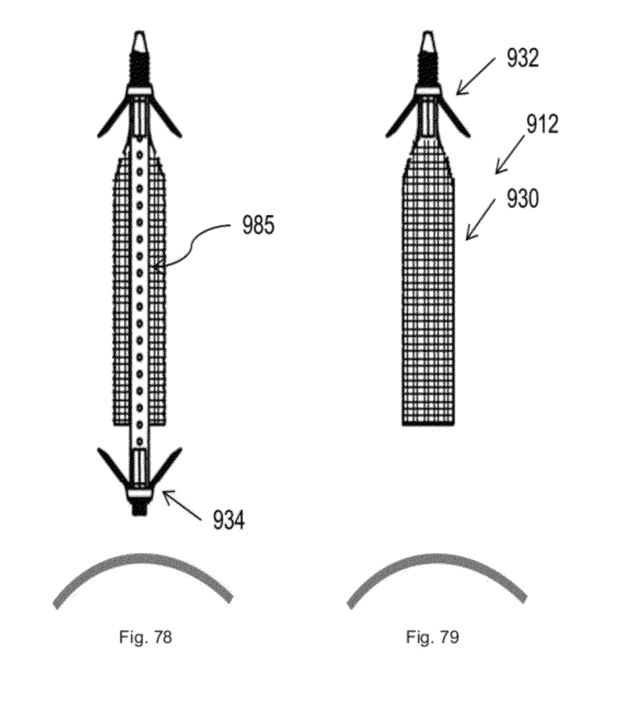

Having described different structural aspects of this embodiment, details of its use will now be described. The apparatus 914 is used to insert the needle 918 into place, the retainer 920 is removed, and the released distal-anchor 932, proximal anchor 934, and mesh 930 spring out laterally into their deployed positions (FIGS. 72-75). When providing glue anchor spots, a glue dispenser is inserted into the needle 918 (if this has not yet been done) and is then actuated to deliver the glue through the needle's glue ports and onto the mesh where they form glues spots that dry or otherwise cure until they bind the mesh to tissue (not shown). Then the needle 918 is rotated and distally advanced to in turn rotate and distally advance the rotary element of the distal anchor 932, while the barbs 938 of the securing element of the distal anchor remain in place, to distally advance the distal end of the mesh to tension the mesh (FIGS. 76-77). Finally, the needle 918 is reverse-rotated to release it from the distal anchor 932 and proximally retracted to withdraw it from the subject's body, and the implant 912 is left in place until the connector shaft 985 and the distal anchor 934 are absorbed by the body, leaving the mesh 930 and the distal anchor 932 tensioned in place in a sling-like fashion (FIGS. 78-79).

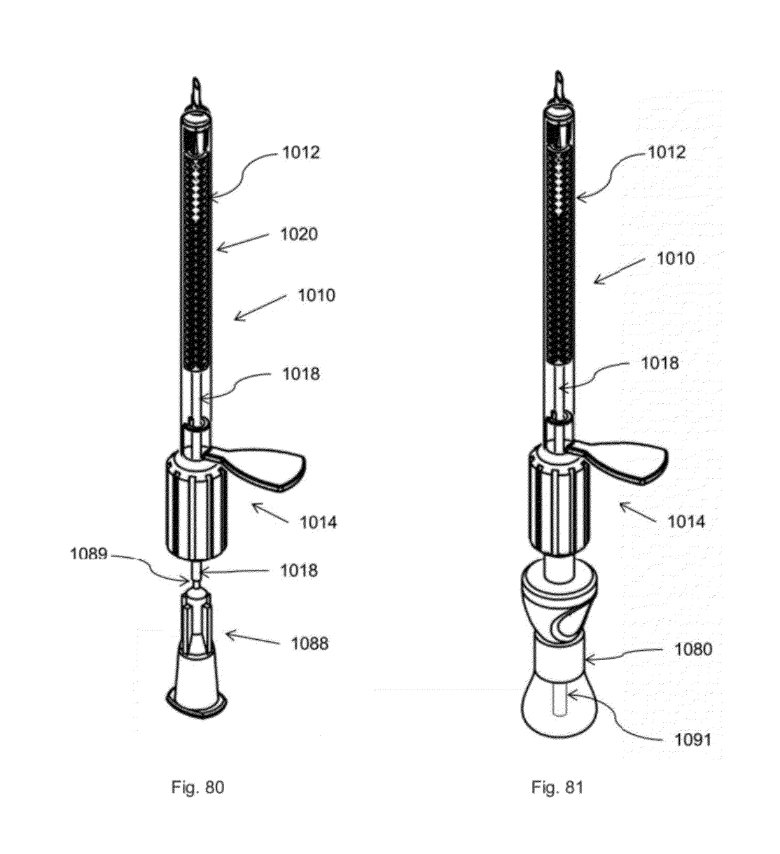

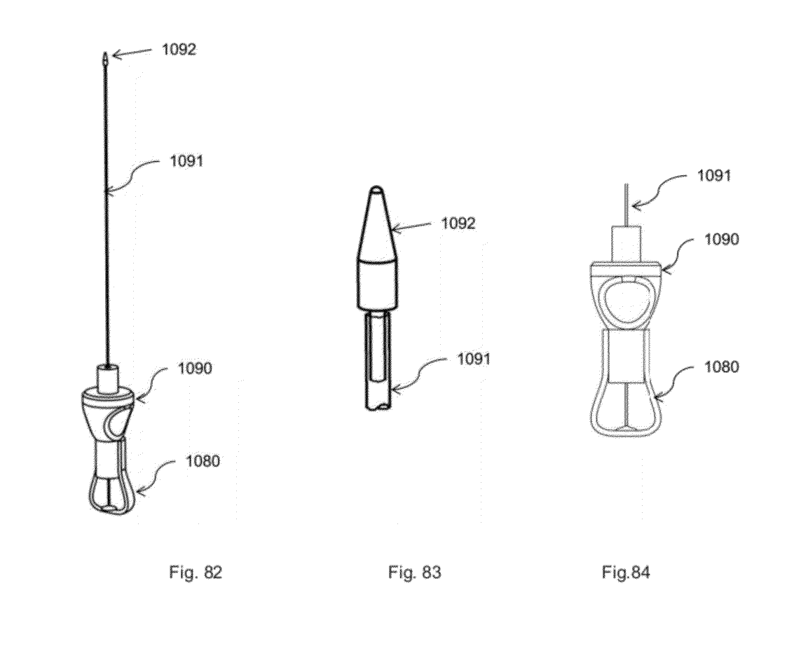

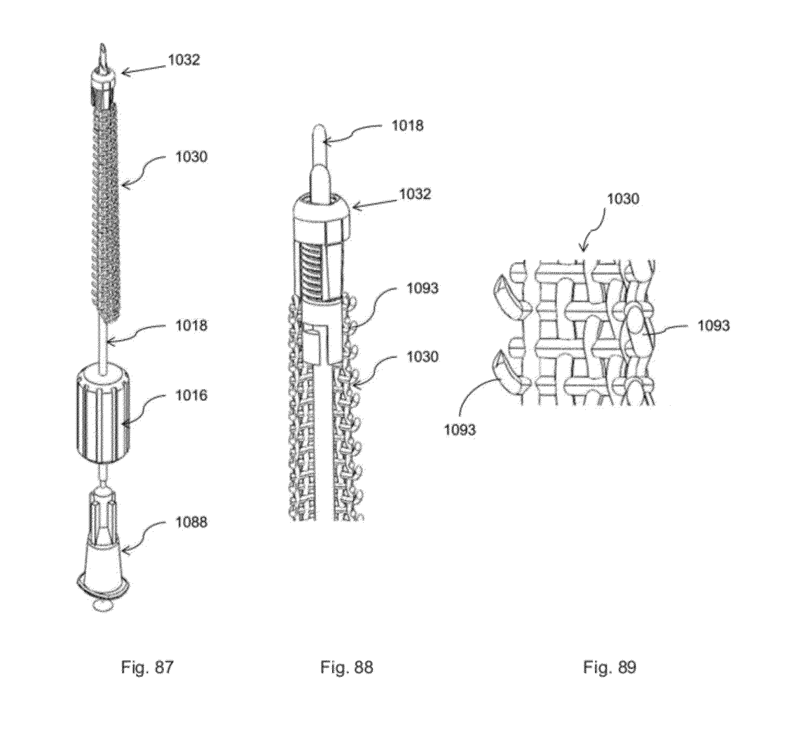

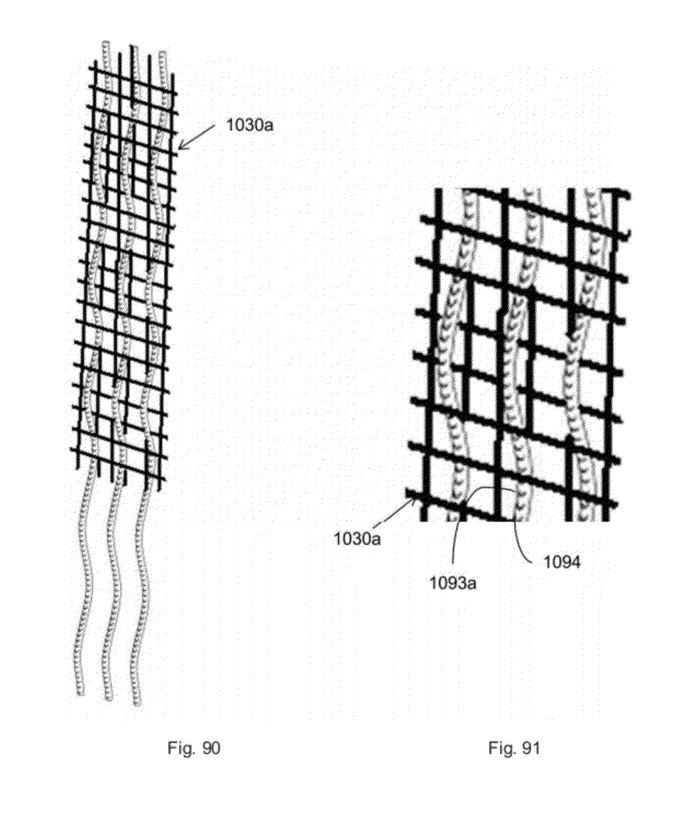

FIGS. 80-93 show a surgical system 1010 according to an eleventh example embodiment of the present invention. The surgical system 1010 and method are the same or similar to those of the other herein-described embodiments, with a few exceptions. As such, the surgical system 1010 includes a surgical implant 1012 and a surgical apparatus 1014 for implanting the surgical implant through the subject's skin and into their body. The apparatus 1014 includes a handle 1016, a needle 1018 extending therefrom, and a retainer 1020 fitted onto the needle. The implant 1014 includes a mesh 1030 and a distal anchor 1032 and mesh-tensioning assembly (e.g., a rotationally adjustable mesh-tensioning anchor as in the two immediately preceding embodiments).

Referring to FIGS. 80-84, in this embodiment a glue-reservoir dispenser 1080 is provided with a handle 1090 attached to it and a glue needle 1091 extending from it. The glue needle 1091 is received within the lumen of the apparatus needle 1018 in a coaxial manner and it has lateral ports (not shown) through which the glue is delivered into the apparatus-needle lumen, out through the apparatus-needle glue ports (not shown), and onto the mesh 1030. The distal end of the glue needle 1091 is of an oburator design, so it is closed off for example by a plug 1092. The glue-reservoir dispenser 1080 can be made of a silicon or other resiliently flexible material.

In addition, a syringe 1088 with a needle 1089 is provided for delivering a medication such as anesthesia to the surgical implantation site. The syringe needle 1089 can be inserted into the lumen of the needle 1018 in a coaxial manner. This embodiment is particularly (but not only) useful when in embodiments in which the mesh 1030 is glued in place and the apparatus needle 1018 includes lateral glue ports. Thus, after inserting the needle 1018 to the surgical implantation site, the syringe 1088 is inserted to deliver anesthesia, and then it is removed and the glue needle is inserted in its place to deliver mesh-anchoring glue.

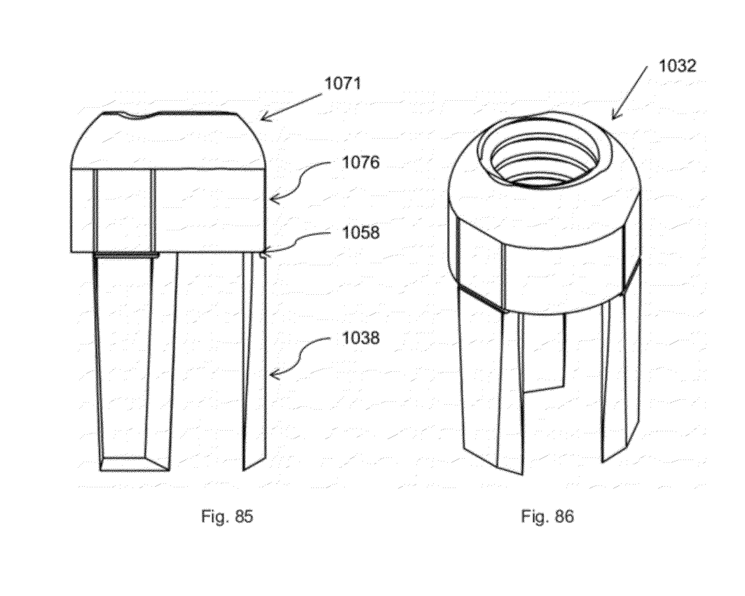

Referring to FIGS. 85-86, in this embodiment the rotary element 1071 of the distal anchor 1034 has a modified design. In particular, it includes modified notches 1058 where the barbs 1038 extend from the threaded collar body 1076 to limit the radially outward travel of the barbs when deployed.

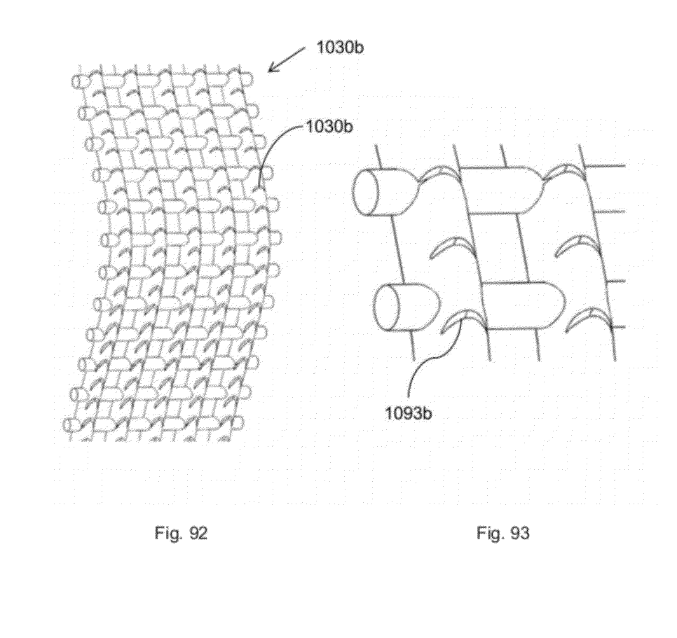

And referring to FIGS. 87-93, the mesh 1030 can include anchoring barbs that help secure it in place. In the embodiment of FIGS. 87-89, the mesh 1030 includes barbs 1093 in the form of hooked threads extending from the mesh. For example, the mesh 1030 can be made of longitudinal and lateral monofilament threads, with the ends of the lateral threads forming the hooks 1093. In the embodiment of FIGS. 90-91, the mesh 1030a is provided without barbs, but barbed sutures 1094a are woven through the mesh so that, when the mesh is tensioned, the sutures are held in place relative to the mesh. The barbed sutures 1094a include integrally formed barbs 1093a and can provided by conventional barbed sutures of a type that is well known and commercially available. And in the embodiment of FIGS. 92-93, the mesh 1030b is provided with integral barbs 1093b of the same type as is common in conventional barbed sutures.

In summary, in use in a female subject, any of the surgical apparatus described herein and shown in the attached drawings can be used to deliver and secure and anchor into place one of the surgical meshes described herein and shown in the attached drawings. The needle tip of the apparatus is inserted through the vaginal skin into the periurethral region and is directed upwards towards the obturator internus muscle or urogential diaphragm. In some embodiments, as the apparatus is advanced, local anesthetic can be delivered through the needle lumen and tip. Once the tip reaches the desired tissue (e.g., muscle or ligament), the tip penetrates that tissue and the barbs of the distal anchor of the surgical mesh are deployed into the tissue for securement. The retainer tube surrounding the needle and mesh is withdrawn by pulling on the detachment tab to expose/deploy the mesh in the periurethral region. By manually pushing up on the vaginal fornix, the distal anchor can be set. The needle is then removed from the body. The mesh can be positioned and tensioned as desired by the practitioner (such as by pulling a tensioning string or rotating a threaded distal anchor). Optionally, an epoxy (e.g., a glue or other adherent substance) can be dispensed through the needle to help set the mesh in place and form one or more anchor points. If the mesh comprises a proximal anchor, the proximal anchor can be manipulated into place and locked into the tissue (such as behind the vaginal skin). If the proximal anchor includes a proximal disc, the disc is positioned outside the vaginal skin as the apparatus is advanced into the body and the disc is later removed from the vagina (e.g., by bio-absorbable sutures). In embodiments with the tensioning string, it can be left in the vagina after the mesh is implanted so that a practitioner can later tighten the mesh sling, if needed, after placement.

It is to be understood that this invention is not limited to the specific dimensions, devices, methods, conditions, or parameters described and/or shown herein, and that the terminology used herein is for the purpose of describing particular embodiments by way of example only. Thus, the terminology is intended to be broadly construed and is not intended to be unnecessarily limiting of the claimed invention. For example, as used in the specification including the appended claims, the singular forms “a,” “an,” and “one” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein.

While the invention has been described with reference to preferred and example embodiments, it will be understood by those skilled in the art that a variety of modifications, additions and deletions are within the scope of the invention, as defined by the following claims.

What is claimed is:

1. A system for treating urinary incontinence in a subject's body, comprising:
a surgical implant including a mesh and a distal anchor coupled to the mesh; and
a surgical device operable for implanting the implant, the device including a needle adapted to be inserted into the subject's body, a handle from which the needle extends and can be grasped, and a retainer that holds the mesh to the needle during insertion and after insertion can be removed from the needle to deploy the mesh to a deployed position for use with the distal anchor securing the mesh in place.

2. The system of claim 1, further comprising a mesh-tensioning assembly adapted to distally move a distal end of the mesh to tension the mesh.

3. The system of claim 2, wherein the mesh-tensioning assembly includes a tensioning string extending distally from the mesh, slidingly engaging the distal anchor, and extending proximally toward the handle, wherein proximally pulling on the tensioning string pulls distally on the mesh to tension the mesh.

4. The system of claim 3, wherein the tensioning string includes unidirectionally advancing mechanical stops and the distal anchor includes an opening through which the tensioning string extends, with the anchor opening and the mechanical stops configured to provide unidirectional advancement of the tensioning string to selectively lock the tensioning string in place.

5. The system of claim 2, wherein the mesh-tensioning assembly is incorporated into the distal anchor and includes a rotary element and a securing element, the rotary element including an anchor body that is mounted to the needle for co-rotation therewith and that is coupled to the mesh, and the securing element including anchor barbs that engage and are held in place in the subject's body, wherein upon rotation of the needle in a first rotary direction the anchor body rotates therewith and the anchor barbs do not to cause the anchor body to advance distally to tension the mesh.

6. The system of claim 5, wherein the anchor body includes screw threads and the securing element includes a collar from which the barbs extend and that has screw threads that mate with the anchor body threads to cause the distal advancement of the anchor body.

7. The system of claim 5, wherein the anchor body includes a bayonet-fitting slot and the needle includes a bayonet-fitting pin that cooperates with the bayonet-fitting slot and is adapted to cause the anchor body to co-rotate with the needle in the first rotary direction but not in a second opposite rotary direction, and wherein upon rotation in the second rotary direction the needle disengages from the anchor body so that the needle pin can be proximally withdrawn from the anchor-body slot.

8. The system of claim 1, wherein the mesh is made of a resiliently flexible material, is compacted into a stored position with the retainer in place for insertion, and resiliently unfurls and laterally extends to a deployed position upon removal of the retainer.

9. The system of claim 8, wherein the retainer is provided by a tube that fits over the mesh with the mesh compacted into the stored position and that includes a failure zone permitting the retainer to be manipulated from a closed retaining position mounted on the mesh and needle to an open releasing position in which the retainer can be removed from the mesh and needle.

10. The system of claim 1, wherein the mesh includes a laterally extendible base that provides an increased surface area for securing in place.

11. The system of claim 10, wherein the mesh base is dome-shaped.

12. The system of claim 11, wherein the mesh base includes a peripheral free edge and further comprising a plurality of base-deploying strings extending from the free edge, wherein pulling proximally on the base-deploying strings manipulates the base from a stored position in which it is compacted against the needle to a deployed position in which it has the dome shape.

13. The system of claim 11, further comprising a proximal anchor including a disc plate having a convex shape to which the dome-shaped base conforms in the deployed position.

14. The system of claim 1, further comprising a proximal anchor including a disc plate that is positionable externally of the subject's body and coupled to a proximal end of the mesh by a bio-absorbable connector, wherein upon absorption of the connector by the subject's body the disc plate is freed from the subject's body.

15. The system of claim 1, further comprising a proximal anchor and a connector shaft extending between the distal anchor and the proximal anchor, the connector shaft made of a bio-absorbable material so that after the mesh has been integrated into the subject's body then the bio-absorbable connector is absorbed by the subject's body.

16. The system of claim 1, wherein the needle includes a lumen and lateral glue ports, and further comprising a glue dispenser from which glue can be delivered through the needle and the glue ports and onto the mesh.

17. The system of claim 1, wherein the needle includes a lumen, and further comprising a syringe from which a medication can be delivered through the needle and into the subject's body.

18. A method of using the system of claim 1 to treat the urinary incontinence in the subject's body, comprising:
inserting the needle and implant into the subject's body;
removing the retainer from the needle to deploy the mesh;
tensioning the mesh to treat the urinary incontinence;
removing the needle from the subject's body to leave the tensioned mesh.

19. A surgical device for implanting a surgical implant to treat urinary incontinence in a subject's body, the implant including a mesh and a distal anchor coupled to the mesh, the device comprising:
a needle adapted to be inserted into the subject's body;
a handle from which the needle extends and can be grasped; and
a retainer that holds the mesh to the needle during insertion and after insertion can be removed from the needle to deploy the mesh to a deployed position for use with the distal anchor securing the mesh in place.

20. A surgical implant for implanting by a surgical device to treat urinary incontinence in a subject's body, the device including a needle for insertion into the subject's body, a grasping handle from which the needle extends, and a retainer that mounts to the needle, the implant comprising:
a distal anchor that secures in place within the subject's body; and
a mesh that is coupled to the distal anchor, held to the needle by the retainer during insertion into the subject's body, and after insertion and removal of the retainer can be laterally extended to a deployed position for use with the distal anchor securing the mesh in place.
